(19) United States
(12) Patent Application Publication
(10) Pub. No.: US 2010/0112019 A1
(43) Pub. Date: May 6, 2010

(54) METHOD FOR MAKING UP KERATINOUS SUBSTANCES AND KIT FOR THE IMPLEMENTATION OF SUCH A METHOD

(75) Inventor: Ludovic Thevenet, Bourg La Reine (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/514,478

(22) PCT Filed: Dec. 19, 2007
(86) PCT No.: PCT/IB07/55232

§ 371 Date: Dec. 9, 2009

Related U.S. Application Data
(60) Provisional application No. 60/899,882, filed on Feb. 7, 2007.

(30) Foreign Application Priority Data
Dec. 20, 2006 (FR) 0655710

(51) Int. Cl. A61K 8/89; A61K 8/02; A61Q 1/00

(52) U.S. Cl. 424/401; 424/78.02

(57) ABSTRACT

A method for making up a support includes application to the support of at least (a) magnetic substances exhibiting a non-zero magnetic susceptibility, (b) one or more compounds X, (c) one of more compounds Y, with at least one of the compounds X and Y being a silicone compound and the compounds Y and X being capable of reacting together by a hydro silylation reaction in the presence of a catalyst, when they are brought into contact with one another, and (d) at least one catalyst. Application of (a), (b), (c) and (d) may be simultaneous or sequential in any order. Exposure to a magnetic field of at least a portion of the magnetic substances takes place before the interaction between the compounds X and Y is complete.

A subject-matter of the present invention is a method for making up a natural support, such as the skin, superficial body growths or the lips, or an artificial support, such as false nails, and also to a kit for the implementation of this method.

Cosmetics compositions and in particular makeup compositions are generally used to modify the properties of visual appearance of the area where they are applied, such as, for example, the colour, the gloss and/or the transparency. Effects of colour and/or of relief are thus very often associated with cosmetic compositions intended to bring out certain applicational areas, such as, for example, the skin, lips, nails or eyelids.

The publication FR 2 876 011 discloses a makeup method in which magnetic substances are exposed to a magnetic field in order to create novel aesthetic effects.

Compositions comprising magnetic substances, for example magnetic pigments, make it possible, for example, to generate a pattern after application which, although it is physically in two dimensions, is perceived as being in three dimensions.

The magnetic substances can be conveyed in an oily phase, which exhibits the distinctive feature of generally being tacky. In point of fact, this tacky nature has the effect of resulting in cosmetic compositions which are capable of transferring, that is to say of being at least partially deposited, leaving marks, on certain supports with which they may be brought into contact and in particular a glass, a cup, a cigarette, an item of clothing or the skin. The result of this is a mediocre persistence of the film applied.

Furthermore, the appearance of such marks, unacceptable in particular on the necks of blouses, can dissuade some women from using this type of makeup.

In addition, the phenomenon of transfer described above also exhibits the disadvantage of being able to affect the orientation of the magnetic substances present in the composition.

In point of fact, this latter aspect is particularly harmful aesthetically insofar as such a modification in the orientation of the magnetic substances can result in a detrimental change in, and even a disappearance of, the desired effect.

There exists a need to retain the orientation of the magnetic substances over time in order to best maintain the effect obtained.

There also exists a need to improve, if appropriate, the non-transfer and hold properties of cosmetic compositions comprising magnetic substances, without, however, harming the comfort of the user, both during application and during use.

The invention is targeted at meeting all or some of these needs.

According to one of its aspect, among others, a subject-matter of the invention is a method for making up a support, comprising at least:
the application to said support of at least (a) magnetic substances exhibiting a non-zero magnetic susceptibility, (b) one or more compounds X, (c) one of more compounds Y, at least one of the compounds X and Y being a silicone compound and the compounds Y and X being capable of reacting together by a hydrosilylation reaction in the presence of a catalyst, or by a condensation reaction or by a crosslinking reaction in the presence of a peroxide, when they are brought into contact with one another, and (d), if appropriate, at least one catalyst or one peroxide, it being possible for the applications (a), (b), (c) and (d) to be simultaneous or sequential in any order, provided that it is favourable to the interaction of the compounds X and Y, and
the exposure to a magnetic field of at least a portion of the magnetic substances so as to move them and/or to orientate them, this exposure taking place before the interaction between the compounds X and Y is complete, so that their interaction does not excessively conflict with the modification in the orientation and/or with the movement of the magnetic substances under the effect of the magnetic field.

The compounds X and Y considered according to the invention prove to be capable of polymerizing in situ, at atmospheric pressure and ambient temperature, and of forming a biocompatible, nontacky, optionally slightly opalescent, indeed even peelable, film which can provide satisfactory adhesion, satisfactory hold and satisfactory comfort and fix the orientation and/or the position of the magnetic substances.

Systems comprising silicone compounds capable of interacting and of forming, on the completion of their interaction, a polymeric film are described in part in the publications WO 01/96450 and GB 2 407 496 from Dow Corning.

Thus, the compound or compounds X, the compound or compounds Y, the magnetic substances exhibiting a non-zero magnetic susceptibility and, if appropriate, the catalyst or the peroxide, can be applied to the keratinous substances starting from several compositions, the compositions respectively comprising the compound or compounds X, the compound or compounds Y, the magnetic substances exhibiting a non-zero magnetic susceptibility, and, if appropriate, the catalyst or the peroxide, alone or as a mixture, or starting from a single composition comprising the compound or compounds X, the compound or compounds Y, the magnetic substances exhibiting a non-zero magnetic susceptibility, and, if appropriate, the catalyst or the peroxide.

It is also possible to alternately apply, to the keratinous substances, several layers of each of the compositions.

According to another aspect, an applied composition combining the compounds X and Y and, if appropriate, the catalyst or the peroxide, comprises at least one of them in an encapsulated form.

The compounds X and Y can be applied simultaneously or successively, the compound X being, for example, applied before the compound Y or vice versa. It is also possible to apply, for example, a composition obtained by mixing, in an extemporaneous way, a first composition comprising at least the compound X and a second composition comprising at least the compound Y, at least one of the first and second compositions additionally comprising at least magnetic substances exhibiting a non-zero magnetic susceptibility, and at least one of said first and second compositions comprising, additionally, where appropriate, at least one catalyst or one peroxide.

The magnetic substances can thus be applied before or after at least one of the compounds X and Y, or simultaneously with this or with these.

The method according to the invention can comprise, in an exemplary embodiment, the application to the support of at least one first cosmetic composition comprising, in a physiologically acceptable medium, the magnetic substances and at least one compound X, respectively at least one compound Y, the exposure of the magnetic substances to the magnetic field, so as to modify their orientation and/or to move them, and the application, to the first cosmetic composition, of at least one second cosmetic composition comprising, in a physiologically acceptable medium, at least one compound Y, respectively at least one compound X, with at least one of said first and second compositions comprising, additionally, where appropriate, at least one catalyst or one peroxide, the exposure of the magnetic substances to the magnetic field so as to modify their orientation and/or to move them taking place prior or subsequent to the application of the second composition.

In an alternative embodiment of the invention, the method comprises at least the application to the support of at least one first cosmetic composition comprising, in a physiologically acceptable medium, said magnetic substances and the application to the first cosmetic composition of at least one second cosmetic composition comprising, in a physiologically acceptable medium, at least one compound X, at least one compound Y, and, where appropriate, at least one catalyst or one peroxide, the exposure of the magnetic substances to the magnetic field so as to modify their orientation and/or to move them taking place prior or subsequent to the application of the second composition but before the compounds X and Y have completed their interaction.

According to a particular embodiment, the second composition can result from the extemporaneous mixing, before it is applied, of at least two separate products, one comprising at least the compound or compounds X and the other comprising at least the compound or compounds Y, with at least one of the first and second compositions comprising, additionally, where appropriate, at least one catalyst or one peroxide.

In the sense of the invention, notably in the embodiment where the composition is obtained as described above, namely by mixing, in an extemporaneous way, a first composition containing at least compound X and a second composition containing at least compound Y, it is to be understood that the mixture thus formed comprises compounds X and/or Y in a form that has not yet reacted and not exclusively in the form of their reaction product by hydrosilylation in the presence of a catalyst, by condensation and/or by crosslinking in the presence of a peroxide.

Thus, formation of the reaction product according to the invention can either be carried out directly on the surface of the keratinous substance that is to be treated, or initiated just before application by extemporaneous mixing of compounds X and Y in conditions favourable for their interaction, formation of the reaction product being in the latter case finalized on the surface of the keratinous substance.

For obvious reasons, and in view of the great reactivity of compounds X and/or Y, it is in fact necessary that their application should be carried out in conditions that are favourable for the manageability of the composition containing it (or them) notably with respect to its spreading, for example. The method according to the invention therefore employs a composition containing compounds X and Y, and therefore not congealed in the form of the expected final film resulting from reaction of all of X and/or of all of Y.

In another alternative embodiment of the invention, the method comprises at least the application to the support of at least one first cosmetic composition comprising, in a physiologically acceptable medium, at least one compound X, respectively at least one compound Y, followed by the application, to the first composition, of at least one second composition comprising, in a physiologically acceptable medium, at least one compound Y, respectively at least one compound X, and said magnetic substances, with at least one of said first and second compositions comprising, additionally, where appropriate, at least one catalyst or one peroxide.

The exposure of the magnetic substances to the magnetic field, so as to modify their orientation and/or to move them, can take place subsequent to the application of the second composition, but before the compounds X and Y have completed their interaction.

In another exemplary embodiment of the invention, the process comprises at least the application to the support of at least one first cosmetic composition comprising, in a physiologically acceptable medium, said magnetic substances, followed by the application, to the first composition, of at least one second cosmetic composition comprising, in a physiologically acceptable medium, at least one compound X, respectively at least one compound Y, and the application, to the second cosmetic composition, of at least one third cosmetic composition comprising, in a physiologically acceptable medium, at least one compound Y, respectively at least one compound X, with at least one of the compositions comprising, additionally, where appropriate, at least one catalyst or one peroxide.

The exposure of the magnetic substances to a magnetic field, so as to modify their orientation and/or to move them, can take place prior or subsequent to the application of the third composition but before the compounds X and Y have completed their interaction.

In another exemplary embodiment of the invention, the method comprises at least the application to said support of at least one first cosmetic composition comprising, in a physiologically acceptable medium, at least one compound X, respectively at least one compound Y, followed by the application, to the first composition, of at least one second cosmetic composition comprising, in a physiologically acceptable medium, said magnetic substances, and the application, to the second composition, of at least one third cosmetic composition comprising, in a physiologically acceptable medium, at least one compound Y, respectively at least one compound X, the second composition being capable of allowing the compounds X and Y to interact, with at least one of the compositions comprising, additionally, where appropriate, at least one catalyst or one peroxide.

The exposure of the magnetic substances to the magnetic field, so as to modify their orientation and/or to move them, can take place, for example, prior or subsequent to the application of the third composition but before the compounds X and Y have completed their interaction.

A further subject-matter of the invention, according to another of its aspects, is a makeup method comprising the steps consisting in:
forming a first deposited layer comprising, in at least one cosmetically acceptable medium, magnetic substances and the compounds X and Y, and, where appropriate, at least one catalyst or peroxide,
exposing the magnetic substances to a magnetic field in order to modify their orientation and/or to move them before the compounds X and Y have completed their interaction,
applying a new deposited layer comprising, in at least one cosmetically acceptable medium, magnetic substances optionally different from those of the first deposited layer and optionally compounds X and Y, and, where appropriate, at least one catalyst or peroxide,
exposing the magnetic substances of the second deposited layer to a magnetic field before, if appropriate, the compounds X and Y of the second deposited layer have completed their interaction.

According to yet another exemplary embodiment, at least one additional layer of at least one third composition comprising a cosmetically acceptable medium, and preferably at least one film-forming polymer and at least one organic (or oily) or aqueous solvent medium, is applied to the layer or layers of the composition or compositions comprising the compounds X, Y and, where appropriate, at least one catalyst or a peroxide, and the magnetic substances exhibiting a non-zero magnetic susceptibility, in order, for example, to improve the hold and/or the comfort of this/these.

When the magnetic substances bring some colour, a modification in their orientation under the effect of the magnetic field can result in a modification in the appearance of the composition.

When the magnetic substances are moved, the form of the deposited layer comprising them may be found to be affected thereby, which makes it possible to create a relief, for example.

During the application of the composition comprising the magnetic substances, this composition may be unexposed to the magnetic field. The latter can be exerted once the application is carried out.

The magnetic field can be applied so as to form at least one pattern, the latter being, for example, related to the geometry of the field lines.

The invention thus makes it possible to create novel makeup effects with a cosmetic composition by making it possible, for example, to produce patterns in relief or patterns conferring an impression of relief or of trompe-l'oeil or various other patterns, which may or may not be geometrical.

When the application takes place on the eyelashes, the magnetic field can move the composition and lengthen the eyelashes, for example.

The magnetic field can also be applied so as to mould the clearness and/or the colour of an area at least of the face or the body on which the deposited layer has been produced.

For example, when the deposited layer is a foundation, the orientation of the magnetic substances under the effect of the magnetic field makes it possible to modify the clearness of the deposited layer and to thus mould the appearance of the face according to the areas exposed to the magnetic field, in order in particular to produce a makeup of the light/dark type, without a sudden transition, if desired, between the light regions and the dark regions. The magnetic field can, for example, be applied so as to darken the sides of the face in order to make it appear thinner than it really is.

In an exemplary embodiment of the invention, the layer applied last can make it possible to obtain an effect of depth, of gloss, of smoothing or another effect. This layer can be transparent, coloured or colourless.

A layer applied before that comprising the magnetic substances can make it possible, for example, to comprise a colouring agent in order to create a coloured background or at least one component targeted at improving the hold of the layers deposited on top and/or the comfort.

The magnetic field can be applied until a fixed appearance of the magnetic substances and in particular of the deposited layer is obtained, that is to say that the appearance of the deposited layer ceases to change even if the magnetic field endures. In an alternative form, the magnetic field can be applied for a period of time which is less than that which causes the definitive orientation and/or the definitive movement of all the magnetic substances of the exposed region.

When the clearness and/or colour of the deposited layer gradually change under the effect of the magnetic field, the user can stop subjecting the magnetic substances to the field when the deposited layer exhibits the desired appearance.

In an exemplary embodiment of the invention, the magnetic field is exerted through a magnetic sheet. Depending on the form of the latter, the field lines will have different geometries, which makes it possible, for example, to increase the number of patterns capable of being produced with the same magnet.

The magnetic field can be exerted successively on different regions of the support coated with the compositions.

The magnetic field can be exerted on unconnected regions of the support in order, for example, to create separate patterns.

A region at least of the support coated with the deposited layer may be unexposed to the magnetic field, so as not to modify the appearance of the deposited layer in this region.

Two regions of the support can be exposed to the magnetic field in an unequal manner.

The composition or compositions applied to the support can be applied in various ways, for example using a cosmetic applicator, preferably a nonmagnetic cosmetic applicator, for example chosen from fine brushes, flocked tips, foams, woven fabrics, nonwoven fabrics, coarse brushes or combs, or without an applicator, the composition or compositions being, for example, spread with the fingers or sprayed. Spraying can be carried out by virtue of a piezoelectric, electrostatic device or by virtue of an airbrush.

In an exemplary embodiment of the invention, at least the composition comprising the magnetic substances is applied to the support through an openwork mask. This can make it possible, for example, to produce a predetermined pattern corresponding to the form of the openwork.

The magnetic substances can be provided in various forms.

Magnetic Substances

The expression “magnetic substances” should not be understood in a limiting manner and covers particles, fibres or agglomerates of particles and/or of fibres, of all shapes, exhibiting a non-zero magnetic susceptibility.

The concentration of magnetic substances in the deposited layer is, for example, between approximately 0.05% and approximately 50% by weight, in particular between approximately 0.1% and approximately 40% by weight, better still between approximately 1% and approximately 30% by weight.

The composition applied can comprise magnetic fibres or other nonspherical substances, such as chains of particles or of fibres.

Preferably, the magnetic substances do not exhibit residual magnetization in the absence of a magnetic field.

The magnetic substances can comprise any magnetic material exhibiting a sensitivity to the lines of a magnetic field, whether this field is produced by a permanent magnet or results from induction, this material being, for example, chosen from nickel, cobalt, iron, their alloys and oxides, in particular Fe₃O₄, and also gadolinium, terbium, dysprosium, erbium, their alloys and oxides. The magnetic material can be of “soft” or “hard” type. The magnetic material can in particular be soft iron.

The magnetic substances may or may not exhibit a multilayer structure, comprising at least one layer of a magnetic material, such as, for example, iron, nickel, cobalt, their alloys and oxides, in particular Fe₃O₄.

The magnetic substances are preferably nonspherical, for example exhibiting an elongated shape. Thus, when these substances are subjected to the magnetic field, they tend to become orientated with their longitudinal axis in the alignment of the field lines and they undergo a change in orientation which is reflected by a change in appearance of the composition.

When the magnetic substances are substantially spherical particles, their appearance is preferably nonhomogenous, so that a change in orientation brings about a change in appearance.

The size of the substances, whatever their shape, is, for example, between 1 nm and 10 mm, better still between 10 nm and 5 mm, even better still between 100 nm and 1 mm, for example between 0.5 μm and 300 μm or 1 μm and 150 μm. The size is that given by the random distribution at half of the population, referred to as D50.

When the substances are particles not having an elongated shape or having an elongated shape with a fairly low aspect ratio, the size of the particles is, for example, less than 1 mm.

The magnetic substances are, for example, composite or noncomposite magnetic pigments.

Magnetic Pigments

Pigments which are very particularly suitable are pearlescent agents comprising iron oxide Fe₃O₄. Pigments exhibiting magnetic properties are, for example, those sold under the trade names Colorona Blackstar Blue, Colorona Blackstar Green, Colorona Blackstar Gold, Colorona Blackstar Red, Cloissoné Nu Antique Super Green, Microna Matte Black (17437), Mica Black (17260), Colorona Patina Silver (17289) and Colorona Patina Gold (117288) from Merck or alternatively Flamenco Twilight Red, Flamenco Twilight Green, Flamenco Twilight Gold, Flamenco Twilight Blue, Timica Nu Antique Silver 110 AB, Timica Nu Antique Gold 212 GB, Timica Nu-Antique Copper 340 AB, Timica Nu Antique Bronze 240 AB, Cloisonné Nu Antique Green 828 CB, Cloisonné Nu Antique Blue 626 CB, Gemtone Moonstone G 004, Cloisonné Nu Antique Red 424 CB, Chroma-Lite Black (4498), Cloissoné Nu Antique Rouge Flambe (code 440 XB), Cloisonné Nu Antique Bronze (240 XB), Cloisonné Nu Antique Gold (222 CB) and Cloissoné Nu Antique Copper (340 XB) from Engelhard.

Mention may be made, as further example of a magnetic pigment capable of being included in the formulation of the composition, of particles of black iron oxide, for example those sold under the name Sicovit Black E172 by BASF.

The magnetic pigments can also comprise iron metal, in particular passivated soft iron, for example obtained from iron carbonyl by employing the process described in U.S. Pat. No. 6,589,331, the content of which is incorporated by reference. These particles can comprise a layer of a surface oxide.

Magnetic Fibres

The term “fibres” denotes generally elongated substances exhibiting, for example, an aspect ratio ranging from 3.5 to 2500 or from 5 to 500, for example from 5 to 150. The aspect ratio is defined by the ratio L/D, where L is the length of the fibre and D is the diameter of the circle within which the greatest transverse cross section of the fibre is framed.

The transverse cross section of the fibres can be framed, for example, within a circle having a diameter ranging from 2 nm to 500 μm, for example ranging from 100 nm to 100 μm, indeed even from 1 μm to 50 μm.

The fibres can, for example, exhibit a length ranging from 1 μm to 10 mm, for example from 0.1 mm to 5 mm, indeed even from 0.3 mm to 3.5 mm.

The fibres can exhibit a weight ranging, for example, from 0.15 to 30 denier (weight in grams per 9 km of yarn), for example from 0.18 to 18 denier.

The fibres can have any shape in transverse cross section, for example circular or polygonal, in particular square, hexagonal or octagonal.

The deposited layer produced on the support to be made up can comprise solid or hollow fibres which are independent or connected to one another, for example plaited.

The deposited layer can comprise fibres having ends which are blunted and/or rounded, for example by polishing.

The fibres may not experience a substantial modification in their shape when they are introduced into the corresponding composition, for example being initially rectilinear and sufficiently rigid to retain their shape. In an alternative form, the fibres may exhibit a flexibility which allows them to substantially change shape in the composition.

The fibres can comprise a non-zero content, which can range up to 100%, of a magnetic material chosen from soft magnetic materials, hard magnetic materials, in particular based on iron, on zinc, on nickel, on cobalt, or on manganese and their alloys and oxides, in particular Fe₃O₄, rare earths, barium sulphate, iron-silicon alloys, optionally comprising molybdenum, Cu₂MnAl, MnBi, or a mixture of these, this list not being limiting.

When the deposited layer comprises fibres comprising magnetic particles, the latter can be present, for example, at least at the surface of the fibre, indeed even at the surface of the fibres only, inside the fibre only or also be dispersed within the fibre in a substantially homogenous fashion.

The fibres can, for example, comprise a nonmagnetic core with a plurality of magnetic particles at its surface.

The fibres can also comprise a synthetic matrix comprising a plurality of magnetic grains dispersed within it.

If appropriate, a synthetic material charged with magnetic particles can itself be coated with a nonmagnetic shell. Such a shell constitutes, for example, a barrier which insulates the magnetic material or materials from the ambient medium and/or can introduce colour. The fibres can comprise a monolithic magnetic core and be coated with a nonmagnetic shell, or the reverse case may apply.

The deposited layer can comprise fibres produced by extrusion or coextrusion of one or more polymeric materials, in particular thermoplastic and/or elastomers. One of the materials extruded can comprise a charge of dispersed magnetic particles.

The fibre can comprise a synthetic material chosen from polyamides, PET, acetates, polyolefins, in particular PE or PP, PVC, polyester-block-amide, plasticized Rilsan®, elastomers, in particular polyester elastomers, PE elastomers, silicone elastomers, or nitrile elastomers, or a mixture of these materials, this list not being limiting.

The deposited layer can comprise composite fibres comprising a magnetic core at least partially coated with at least one synthetic or natural nonmagnetic material. The coating of the magnetic core can be carried out, for example, by coextrusion, around the core, of a shell made of a nonmagnetic material.

The coating of the core can also be carried out in another way, for example by in situ polymerization.

The core can be monolithic or can comprise a charge of magnetic grains dispersed in a matrix.

The deposited layer can also comprise composite fibres obtained by coating a synthetic or natural nonmagnetic core with a synthetic material charged with magnetic particles, the core being composed, for example, of a fibre made of wood, rayon, polyamide, a plant material, polyolefin, in particular polyethylene, Nylon®, polyimideamide or aramid, this list not being limiting.

The deposited layer can also comprise magnetic composite particles, in particular a magnetic latex.

Magnetic Composite Particles

A magnetic composite particle is a composite material composed of an organic or inorganic matrix and of magnetic grains. The magnetic composite particles can thus comprise grains of a magnetic material at their surface and/or within them. The composite particles can be composed of a magnetic core coated with an organic or inorganic matrix, or vice versa.

The magnetic composite particles comprise, for example, one of the abovementioned magnetic materials.

The size of the magnetic composite particles is, for example, between 1 nm and 1 mm, better still between 100 nm and 500 μm, even better still between 500 nm and 100 μm. The term “size” denotes the size given by the random particle size distribution at half the population, referred to as D50.

The thesis by C. Goubault, 23 Mar. 2004, incorporated here by reference, gives, in chapter 1, a restatement of the state of the art with regard to magnetic composite particles and draws up a list of preparation processes which can be used to prepare magnetic composite particles, namely a separate synthesis of the magnetic grains and of the matrix, a synthesis of the magnetic grains in contact with the matrix or a synthesis of the matrix in the presence of the magnetic grains.

Magnetic composite particles with an inorganic matrix composed of silica are available commercially from Kisker. Magnetic composite particles with an organic matrix also capable of being used in the invention are provided by Dynal, Seradyn, Estapor and Ademtech.

More particularly, magnetic latex composed of ferrite grains uniformly distributed in a polystyrene matrix, this latex comprising 65% of iron oxide, the mean diameter of the polystyrene particles being 890 nm, and the content by weight on a dry basis being 10%, are available commercially from Estapor under the reference M1-070/60.

Ferrofluid

The composition can comprise a ferrofluid, that is to say a stable colloidal suspension of magnetic particles, in particular of magnetic nanoparticles.

The particles, with a size, for example, of the order of a few tens of nanometres, are dispersed in a solvent (water, oil, organic solvent), either using a surfactant or dispersing agent or by electrostatic interactions.

The ferrofluids are, for example, prepared by grinding ferrites or other magnetic particles until nanoparticles are obtained, which nanoparticles are subsequently dispersed in a fluid comprising a surfactant, which surfactant is adsorbed on the particles and stabilizes them, or by precipitation in a basic medium from a solution of metal ions.

Each particle of the ferrofluid exhibits a magnetic moment determined by the size of the particle and by the nature of the magnetic material.

Under the action of a magnetic field, the magnetic moments of the particles tend to become aligned along the field lines, with appearance of a non-zero magnetization in the liquid. If the field is cancelled, there is no hysteresis and the magnetization is reduced to zero.

Above a threshold field value, it is also possible to cause macroscopic changes in the liquid, for example the appearance of peaks or a modification in the rheological properties.

The name “ferrofluid” also encompasses an emulsion of droplets of ferrofluid in a solvent. Each drop then comprises colloidal magnetic particles in stable suspension. This makes it possible to have available a ferrofluid in any type of solvent. The size of the magnetic particles in suspension in the ferrofluid is, for example, between 1 nm and 10 μm, better still between 1 nm and 1 μm, even better still between 1 nm and 100 nm. The term “size” denotes the size given by the random particle size distribution at half the population, referred to as D50.

Mention may in particular be made of the ferrofluids sold by Liquids Research Ltd under the references:
WHKS1S9 (A, B or C), which is an aqueous-based ferrofluid comprising magnetite (Fe₃O₄), having particles with a diameter of 10 nm.
WHJS1 (A, B or C), which is a ferrofluid based on isoparaffin and on magnetite (Fe₃O₄) particles with a diameter of 10 nm.
BKS25 dextran, which is an aqueous-based ferrofluid stabilized by dextran, comprising magnetite (Fe₃O₄) particles with a diameter of 9 nm.

Chains of Magnetic Particles and/or Fibres

The deposited layer can comprise agglomerates of particles or fibres, the greatest size of which, for example the length, is, for example, between 1 nm and 10 mm, for example between 10 nm and 5 mm, or between 100 nm and 1 mm, or also between 0.5 μm and 3.5 mm, for example between 1 μm and 150 μm. The size denotes that given by the random particle size distribution at half the population, referred to as D50.

Chains of magnetic particles can be obtained, for example, by collecting together colloidal magnetic particles, as is described in the publications “Permanently linked monodisperse paramagnetic chains”, E. M. Furst, C. Suzuki, M. Fermigier and A. P. Gast, Langmuir, 14, 7334-7336 (1998), “Suspensions de particules magnétiques”, M. Fermigier and Y. Grasselli, Bulletin de la SFP (105) July 96, and “Flexible magnetic filaments as micromechanical sensors”, C. Goubault, P. Jop, M. Fermigier, J. Baudry, E. Bertrand and J. Bibette, Phys. Rev. Lett., 91, 26, 260802-1 to 260802-4 (2003), the contents of which are incorporated by reference.

These papers describe in particular how to proceed in order to obtain chains of magnetic latex particles, comprising a polystyrene matrix comprising iron oxide grains, which particles are functionalized at the surface and are permanently bonded to one another following a chemical reaction, in particular via covalent bonds between the surfaces of the adjacent particles; a description is also given of a process for obtaining chains of droplets of ferrofluid emulsion bonded to one another via interactions of a physical nature. The length and the diameter of the permanent chains thus obtained can be controlled. Such magnetic chains constitute anisotropic magnetic objects which can be orientated and moved under the effect of a magnetic field.

The sizes of the magnetic chains can correspond to the same conditions as the magnetic fibres.

Compounds X and Y

Silicone compound means a polyorganosiloxane compound, i.e. comprising at least two organosiloxane units, for example at least 5 organosiloxane units, notably at least 10 organosiloxane units. According to a particular embodiment, at least one of compounds X and Y, or compounds X and compounds Y are silicone compounds. Compounds X and Y can be aminated or non-aminated.

According to another embodiment, at least one of compounds X and Y is a polymer whose main chain is formed primarily of organosiloxane units. Among the silicone compounds mentioned below, some may display both film-forming and adhesive properties, depending for example on their proportion of silicone or depending on whether they are used mixed with a particular additive. It is therefore possible to adjust the film-forming properties or the adhesive properties of said compounds according to the proposed use, which is the case in particular for the so-called “room temperature vulcanization” reactive elastomeric silicones.

Compounds X and Y can react with each other at a temperature varying between room temperature and 180° C. Advantageously, compounds X and Y are capable of reacting together at room temperature (20±5° C.) and atmospheric pressure, or advantageously in the presence of a catalyst, by a hydrosilylation reaction or a condensation reaction, or a crosslinking reaction in the presence of a peroxide.

Polar Groups

According to a particular embodiment, at least one of compounds X and Y, for example compound X, bears at least one polar group that is able to form at least one hydrogen bond with keratinous substances.

By polar group, we mean a group having carbon atoms and hydrogen atoms in its chemical structure and at least one heteroatom (such as O, N, S and P), such that said group is able to establish at least one hydrogen bond with keratinous substances.

Compounds bearing at least one group that can form a hydrogen bond are particularly advantageous, as they endow the compositions containing them with better adherence on keratinous substances.

The polar group or groups borne by at least one of compounds X and Y is/are able to establish a hydrogen bond, and include either a hydrogen atom bound to an electronegative atom, or an electronegative atom for example an oxygen, nitrogen or sulphur atom. When the group has a hydrogen atom bound to an electronegative atom, the hydrogen atom can interact with another electronegative atom borne for example by another molecule, such as keratin, to form a hydrogen bond. When the group has an electronegative atom, the electronegative atom can interact with a hydrogen atom bound to an electronegative atom borne for example by another molecule, such as keratin, to form a hydrogen bond.

Advantageously, these polar groups can be selected from the following groups:
carboxylic acids —COOH,
alcohols, such as: —CH₂OH or —CH(R)OH, R being an alkyl radical having from 1 to 6 carbon atoms,
amino of formula —NR₁R₂, in which R₁ and R₂, which may be identical or different, represent an alkyl radical having from 1 to 6 carbon atoms or one of R₁ or R₂ denotes a hydrogen atom, and the other one of R₁ and R₂ represents an alkyl radical having from 1 to 6 carbon atoms,
pyridino,
amido of formula —NH—COR′ or —CO—NH—R′ in which R′ represents a hydrogen atom or an alkyl radical having from 1 to 6 carbon atoms,
pyrrolidino preferably selected from the groups of formula:

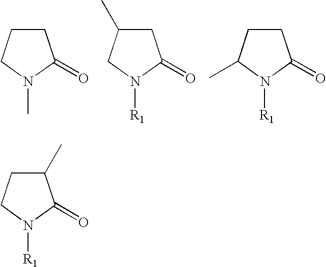

R₁ being an alkyl radical having from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulphonamido such as —NR′—S(═O)₂—R′, R′ corresponding to the above definition.

Preferably, these polar groups are present at a content less than or equal to 10 wt. % relative to the weight of each compound X or Y, preferably less than or equal to 5 wt. %, for example at a content ranging from 1 to 3 wt. %.

The polar group or groups can be located in the main chain of compound X and/or Y or can be pendant from the main chain or located at the ends of the main chain of compound X and/or Y.

1—Compounds X and Y Capable of Reacting by Hydrosilylation

According to one embodiment, the invention relates to a method for making up a support comprising at least:
the application to said support of at least (a) magnetic substances exhibiting a non-zero magnetic susceptibility, (b) one or more compounds X, (c) one of more compounds Y, with at least one of the compounds X and Y being a silicone compound and said compounds X and Y being capable of reacting together by a hydrosilylation reaction in the presence of a catalyst, when they are brought into contact with one another, and (d) at least one catalyst, it being possible for the applications (a), (b), (c) and (d) to be simultaneous or sequential in any order, provided that it is favourable to the interaction of said compounds X and Y, and
the exposure to a magnetic field of at least a portion of the magnetic substances so as to move them and/or to orientate them, this exposure taking place before the interaction between the compounds X and Y is complete, so that their interaction does not excessively conflict with the modification in the orientation and/or with the movement of the magnetic substances under the effect of the magnetic field.

According to this embodiment, compounds X and Y are capable of reacting by hydrosilylation in the presence of a catalyst, said reaction being represented schematically in a simplified manner as follows:

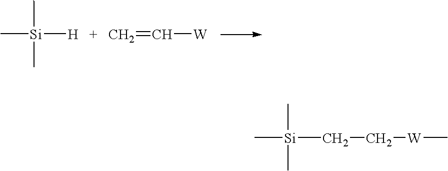

with W representing a carbon chain and/or silicone chain containing one or more unsaturated aliphatic groups.

In this case, compound X can be selected from silicone compounds comprising at least two unsaturated aliphatic groups. As an example, compound X can be a polyorganosiloxane comprising a silicone main chain whose unsaturated aliphatic groups are pendant from the main chain (side group) or located at the ends of the main chain of the compound (end group). These particular compounds will be called, hereinafter, polyorganosiloxanes with unsaturated aliphatic groups.

According to one embodiment, compound X and/or compound Y bear at least one polar group, as described above, capable of forming at least one hydrogen bond with keratinous substances. This polar group is advantageously carried by compound X, which has at least two unsaturated aliphatic groups.

According to one embodiment, compound X is selected from the polyorganosiloxanes comprising at least two unsaturated aliphatic groups, for example two or three vinyl or allyl groups, each attached to a silicon atom.

According to an advantageous embodiment, compound X is selected from the polyorganosiloxanes containing siloxane units of formula:

$${R}_{m} {R}^{′} {SiO}_{( \frac{3 - m}{2} )} \qquad ( I )$$

in which:
R represents a linear or cyclic, monovalent hydrocarbon group, having from 1 to 30 carbon atoms, preferably from 1 to 20, and better still from 1 to 10 carbon atoms, for example a short-chain alkyl radical, comprising for example from 1 to 10 carbon atoms, in particular a methyl radical or alternatively a phenyl group, preferably a methyl radical,
m is equal to 1 or 2 and
R′ represents:
   an unsaturated aliphatic hydrocarbon group having from 2 to 10, preferably from 3 to 5 carbon atoms, for example a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom; we may mention, as group R′, the vinyl and allyl groups and mixtures thereof; or
   an unsaturated cyclic hydrocarbon group having from 5 to 8 carbon atoms, for example a cyclohexenyl group.

Preferably R′ is an unsaturated aliphatic hydrocarbon group, preferably a vinyl group.

According to one embodiment, R represents an alkyl radical having from 1 to 10 carbon atoms or alternatively a phenyl group, and preferably a methyl radical, and R′ is a vinyl group.

According to a particular embodiment, the polyorganosiloxane also contains units of formula:

$${R}_{n} {SiO}_{( \frac{4 - n}{2} )} \qquad ( II )$$

in which R is a group as defined previously, and n is equal to 1, 2 or 3.

According to a variant, compound X can be a silicone resin comprising at least two ethylenic unsaturations, said resin being capable of reacting with compound Y by hydrosilylation in the presence of a catalyst. We may mention for example the resins of type MQ or MT which themselves bear —CH═CH₂ unsaturated reactive end groups.

These resins are crosslinked organosiloxane polymers.

The class of the silicone resins is known by the name “MDTQ”, the resin being described in relation to the different siloxane monomer units that it contains, each of the letters “MDTQ” characterizing a type of unit.

The letter M represents the monofunctional unit of formula (CH₃)₃SiO_{1/2}, the silicon atom being bound to a single oxygen atom in the polymer comprising said unit.

The letter D denotes a bifunctional unit (CH₃)₂SiO_{2/2} in which the silicon atom is bound to two oxygen atoms.

The letter T represents a trifunctional unit of formula (CH₃)SiO_{3/2}.

In units M, D, T defined above, at least one of the methyl groups can be substituted with a group R other than the methyl group, such as a hydrocarbon radical (notably alkyl) having from 2 to 10 carbon atoms or a phenyl group or alternatively a hydroxyl group.

Finally, the letter Q denotes a tetrafunctional unit SiO₄₁₂ in which the silicon atom is bound to four hydrogen atoms which are themselves attached to the rest of the polymer. As examples of said resins, we may mention the MT silicone resins such as poly(phenyl-vinylsilsesquioxane) such as that marketed under the reference SST-3PV1 by the company Gelest.

Preferably, compounds X have from 0.01 to 1 wt. % of unsaturated aliphatic groups.

Advantageously, compound X is selected from the polyorganopolysiloxanes, notably those comprising the siloxane units (I) and optionally (II) described previously.

Compound Y preferably has at least two free Si—H groups (hydrogenosilane groups).

Compound Y can be selected advantageously from the polyorganosiloxanes comprising at least one alkylhydrogenosiloxane unit of the following formula:

$${R}_{p} {HSiO}_{( \frac{3 - p}{2} )} \qquad ( III )$$

in which:

R represents a linear or cyclic, monovalent hydrocarbon group, having from 1 to 30 carbon atoms, for example an alkyl radical having from 1 to 30 carbon atoms, preferably from 1 to 20 and better still from 1 to 10 carbon atoms, in particular a methyl radical, or alternatively a phenyl group and p is equal to 1 or 2. Preferably R is a hydrocarbon group, preferably methyl.

These polyorganosiloxane compounds Y with alkylhydrogenosiloxane units can additionally contain units of formula:

$${R}_{n} {SiO}_{( \frac{4 - n}{2} )} \qquad ( II )$$

as defined above.

Compound Y can be a silicone resin comprising at least one unit selected from the units M, D, T, Q as defined above and comprising at least one Si—H group such as the poly(methyl-hydridosilsesquioxane) marketed under the reference SST-3 MH1.1 by the company Gelest.

Preferably, these polyorganosiloxane compounds Y have from 0.5 to 2.5 wt. % of Si—H groups.

Advantageously, the radicals R represent a methyl group in formulae (I), (II), (III) above.

Preferably, these polyorganosiloxanes Y have end groups of formula (CH₃)₃SiO_{1/2}.

Advantageously, the polyorganosiloxanes Y have at least two alkylhydrogenosiloxane units of formula —(H₃C)(H)SiO— and optionally include —(H₃C)₂SiO— units.

These polyorganosiloxane compounds Y with hydrogenosilane groups are described for example in document EP 0465744.

According to one variant, compound X is selected from the organic oligomers or polymers (by organic, we mean compounds whose main chain is not a silicone chain, preferably compounds not containing silicon atoms) or from hybrid organic/silicone polymers or oligomers, said oligomers or polymers bearing at least 2 unsaturated reactive aliphatic groups, compound Y being selected from the polyorganosiloxanes Y with hydrogenosilane groups mentioned above.

According to one embodiment, the organic or hybrid organic/silicone compounds X bearing at least 2 unsaturated reactive aliphatic groups, have at least one polar group as described above.

Compound X, of organic nature, can then be selected from the vinylic, (meth)acrylic polymers or oligomers, polyesters, polyurethanes and/or polyureas, polyethers, perfluoropolyethers, polyolefins such as polybutene, polyisobutylene, dendrimers or organic hyperbranched polymers, or mixtures thereof.

In particular, the organic polymer or the organic moiety of the hybrid polymer can be selected from the following polymers:

a) polyesters with ethylenic unsaturation(s):

This is a group of polymers of the polyester type having at least 2 ethylenic double bonds, randomly distributed in the main chain of the polymer. These unsaturated polyesters are obtained by polycondensation of a mixture:
of linear or branched aliphatic or cycloaliphatic dicarboxylic acids notably having 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with ethylenic unsaturations such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and/or of diol dimers resulting from reduction of dimers of fatty acids as defined previously, and
of one or more dicarboxylic acids or their anhydrides having at least one polymerizable ethylenic double bond and having from 3 to 50 carbon atoms, preferably from 3 to 20 and better still from 3 to 10 carbon atoms, such as maleic acid, fumaric acid or itaconic acid.

b) polyesters with (meth)acrylate side and/or end groups:

This is a group of polymers of the polyester type obtained by polycondensation of a mixture:
of linear or branched aliphatic or cycloaliphatic dicarboxylic acids notably having from 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with an ethylenic unsaturation such as the dimers of oleic or linoleic acids described in application EP-A-959 066 (paragraph

marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyesters differ from those described above in section a) by the fact that the ethylenic double bonds are not located in the main chain but on side groups or at the end of the chains. These ethylenic double bonds are those of the (meth)acrylate groups present in the polymer.

Such polyesters are marketed for example by the company UCB under the designations EBECRYL® (EBECRYL® 450: molecular weight 1600, on average 6 acrylate functions per molecule, EBECRYL® 652: molecular weight 1500, on average 6 acrylate functions per molecule, EBECRYL® 800: molecular weight 780, on average 4 acrylate functions per molecule, EBECRYL® 810: molecular weight 1000, on average 4 acrylate functions per molecule, EBECRYL® 50 000: molecular weight 1500, on average 6 acrylate functions per molecule).

c) polyurethanes and/or polyureas with (meth)acrylate groups, obtained by polycondensation:
of aliphatic, cycloaliphatic and/or aromatic diisocyanates, triisocyanates and/or polyisocyanates notably having from 4 to 50, preferably from 4 to 30 carbon atoms, such as hexamethylenediisocyanate, isophoronediisocyanate, toluenediisocyanate, diphenylmethanediisocyanate or isocyanurates of formula:

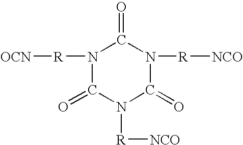

resulting from the trimerization of 3 molecules of diisocyanates OCN—R—CNO, where R is a linear, branched or cyclic hydrocarbon radical having from 2 to 30 carbon atoms;
of polyols, notably of diols, free from polymerizable ethylenic unsaturations, such as 1,4-butanediol, ethylene glycol or trimethylolpropane, and/or of polyamines, notably of aliphatic, cycloaliphatic and/or aromatic diamines, notably having from 3 to 50 carbon atoms, such as ethylenediamine or hexamethylenediamine, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyurethanes/polyureas with acrylate groups are marketed for example under the designation SR 368 (tris(2-hydroxyethyl)isocyanurate-triacrylate) or CRAYNOR® 435 by the company CRAY VALLEY, or under the designation EBECRYL® by the company UCB (EBECRYL® 210: molecular weight 1500, 2 acrylate functions per molecule, EBECRYL® 230: molecular weight 5000, 2 acrylate functions per molecule, EBECRYL® 270: molecular weight 1500, 2 acrylate functions per molecule, EBECRYL® 8402: molecular weight 1000, 2 acrylate functions per molecule, EBECRYL® 8804: molecular weight 1300, 2 acrylate functions per molecule, EBECRYL® 220: molecular weight 1000, 6 acrylate functions per molecule, EBECRYL® 2220: molecular weight 1200, 6 acrylate functions per molecule, EBECRYL® 1290: molecular weight 1000, 6 acrylate functions per molecule, EBECRYL® 800: molecular weight 800, 6 acrylate functions per molecule).

We may also mention the water-soluble aliphatic diacrylate polyurethanes marketed under the designations EBECRYL® 2000, EBECRYL® 2001 and EBECRYL® 2002, and the diacrylate polyurethanes in aqueous dispersion marketed under the trade names IRR® 390, IRR® 400, IRR® 422 IRR® 424 by the company UCB.

d) polyethers with (meth)acrylate groups obtained by esterification, by (meth)acrylic acid, of the hydroxyl end groups of homopolymers or of C₁₋₄ alkylene glycol copolymers, such as polyethylene glycol, polypropylene glycol, copolymers of ethylene oxide and of propylene oxide preferably having a weight-average molecular weight below 10 000, polyethoxylated or polypropoxylated trimethylolpropane.

Di(meth)acrylate polyoxyethylenes of suitable molecular weight are marketed for example under the designations SR 259, SR 344, SR 610, SR 210, SR 603 and SR 252 by the company CRAY VALLEY or under the designation EBECRYL® 11 by UCB. Polyethoxylated trimethylolpropane triacrylates are marketed for example under the designations SR 454, SR 498, SR 502, SR 9035, SR 415 by the company CRAY VALLEY or under the designation EBECRYL® 160 by the company UCB. Polypropoxylated trimethylolpropane triacrylates are marketed for example under the designations SR 492 and SR 501 by the company CRAY VALLEY.

e) epoxyacrylates obtained by reaction between
at least one diepoxide selected for example from:
   (i) bisphenol A diglycidyl ether,
   (ii) a diepoxy resin resulting from the reaction between bisphenol A diglycidyl ether and epichlorohydrin,
   (iii) an epoxyester resin with α,ω-diepoxy end groups resulting from the condensation of a dicarboxylic acid having from 3 to 50 carbon atoms with a stoichiometric excess of (i) and/or (ii),
   (iv) an epoxyether resin with α,ω-diepoxy end groups resulting from the condensation of a diol having from 3 to 50 carbon atoms with a stoichiometric excess of (i) and/or (ii),
   (v) natural or synthetic oils bearing at least 2 epoxide groups, such as epoxidized soya oil, epoxidized linseed oil and epoxidized vernonia oil,
   (vi) a phenol-formaldehyde polycondensate (Novolac® resin), of which the end groups and/or side groups have been epoxidized, and
one or more carboxylic acids or carboxylic polyacids having at least one ethylenic double bond at α,β of the carboxyl group such as (meth)acrylic acid or crotonic acid or esters of (meth)acrylic acid and of a diol or polyol having from 2 to 20 carbon atoms, preferably from 2 to 6 carbon atoms such as 2-hydroxyethyl (meth)acrylate.

Such polymers are marketed for example under the designations SR 349, SR 601, CD 541, SR 602, SR 9036, SR 348, CD 540, SR 480, CD 9038 by the company CRAY VALLEY, under the designations EBECRYL® 600 and EBECRYL® 609, EBECRYL® 150, EBECRYL® 860, EBECRYL® 3702 by the company UCB and under the designations PHOTOMER® 3005 and PHOTOMER® 3082 by the company HENKEL.

f) (C₁₋₅₀ alkyl) poly(meth)acrylates, said alkyl being linear, branched or cyclic, bearing at least two functions with ethylenic double bond carried by the lateral and/or terminal hydrocarbon chains.

Such copolymers are marketed for example under the designations IRR® 375, OTA® 480 and EBECRYL® 2047 by the company UCB.

g) polyolefins such as polybutene, polyisobutylene,

h) perfluoropolyethers with acrylate groups obtained by esterification, for example by (meth)acrylic acid, of perfluoropolyethers bearing hydroxyl side and/or end groups.

These α,ω-diol perfluoropolyethers are described notably in EP-A-1057849 and are marketed by the company AUSIMONT under the designation FOMBLIN® Z DIOL.

i) dendrimers and hyperbranched polymers bearing (meth)acrylate or (meth)acrylamide end groups obtained respectively by esterification or amidation of dendrimers and of hyperbranched polymers with hydroxyl or amino terminal functions, by (meth)acrylic acid.

The dendrimers (from the Greek dendron=tree) are “tree-like” polymer molecules, i.e. highly branched, invented by D. A. Tomalia and his team at the beginning of the 1990's (Donald A. Tomalia et al., Angewandte Chemie, Int. Engl. Ed., Vol. 29, No. 2, pages 138-175). They are structures constructed around a, generally polyvalent, central unit. Branched chain-extending units are arranged according to a perfectly defined structure around this central unit, thus giving rise to symmetrical, monodispersed macromolecules having a well-defined chemical and stereochemical structure. Dendrimers of the polyamidoamine type are marketed for example under the name STARBURST® by the company DENDRITECH.

The hyperbranched polymers are polycondensates, generally of the polyester, polyamide or polyethyleneamine type, obtained from multifunctional monomers, which have a tree-like structure similar to that of the dendrimers but far less regular than the latter (see for example WO-A-93/17060 and WO 96/12754).

The company PERSTORP markets hyperbranched polyesters under the name BOLTORN®. Hyperbranched polyethyleneamines are available under the name COMBURST® from the company DENDRITECH. Hyperbranched poly(esteramide)s with hydroxyl end groups are marketed by the company DSM under the name HYBRANE®.

These dendrimers and hyperbranched polymers, esterified or amidated by acrylic and/or methacrylic acid, differ from the polymers described in sections a) to h) above by the very large number of ethylenic double bonds present. This increased functionality, generally greater than 5, makes them particularly useful in enabling them to act as a “crosslinking node”, i.e. a multiple crosslinking site.

It is therefore possible to use these dendritic and hyperbranched polymers in association with one or more of the above polymers and/or oligomers a) to h).

1a—Additional Reactive Compounds

According to one embodiment, the compositions containing compound X and/or Y can additionally comprise an additional reactive compound such as:
organic or mineral particles having on their surface at least 2 unsaturated aliphatic groups—we may mention for example the silicas surface-treated for example with silicone compounds with vinylic groups such as for example cyclotetramethyltetravinylsiloxane-treated silica,
silazane compounds such as hexamethyldisilazane.

1b—Catalyst

The hydrosilylation reaction takes place in the presence of a catalyst which can be present with one or other of the compounds X or Y or can be present on its own. For example, this catalyst can be present in the composition in an encapsulated form if the two compounds X and Y, which it must cause to interact, are present in this same composition in an unencapsulated form or conversely it can be contained there in an unencapsulated form if at least one of compounds X and Y is present in the composition in an encapsulated form. The catalyst is preferably based on platinum or tin.

We may mention for example platinum-based catalysts deposited on a support of silica gel or of powdered charcoal, platinum chloride, salts of platinum and of chloroplatinic acids.

The chloroplatinic acids are preferably used in hexahydrate or anhydrous form, which are easily dispersible in organosilicone media.

We may also mention platinum complexes, such as those based on chloroplatinic acid hexahydrate and divinyl tetramethyldisiloxane.

The catalyst can be present at a content in the range from 0.0001 to 20 wt. % relative to the total weight of the composition containing it.

Compounds X and/or Y can be combined with polymerization inhibitors or retarders, and more particularly inhibitors of the catalyst. Non-limitatively, we may mention cyclic polymethylvinylsiloxanes, and in particular tetravinyl tetramethyl cyclotetrasiloxane, acetylenic alcohols, preferably volatile, such as methylisobutynol.

The presence of ionic salts, such as sodium acetate, can have an influence on the rate of polymerization of the compounds.

As an example of a combination of compounds X and Y reacting by hydrosilylation in the presence of a catalyst, we may mention the following references offered by the company Dow Corning: DC 7-9800 Soft Skin Adhesive Parts A & B, as well as the combination of the following mixtures A and B prepared by Dow Corning:

| | | | |
|---|---|---|---|
| Ingredient (INCI name) | CAS No. | Contents (%) | Function |

MIXTURE A:

| | | | |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| | | | |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

Advantageously, compounds X and Y are selected from silicone compounds capable of reacting by hydrosilylation in the presence of a catalyst; in particular compound X is selected from the polyorganosiloxanes comprising units of formula (I) described above and compound Y is selected from organosiloxanes comprising alkylhydrogenosiloxane units of formula (III) described above.

According to a particular embodiment, compound X is a polydimethylsiloxane with vinylic end groups, and compound Y is a polymethylhydrogenosiloxane.

2/ Compounds X and Y Capable of Reacting by Condensation

According to one embodiment, the invention relates to a method for making up a support comprising at least:
the application to said support of at least (a) magnetic substances exhibiting a non-zero magnetic susceptibility, (b) one or more compounds X, (c) one of more compounds Y, with at least one of the compounds X and Y being a silicone compound and said compounds X and Y being capable of reacting together by a condensation reaction, when they are brought into contact with one another, and (d) optionally, at least one catalyst, it being possible for the applications (a), (b), (c) and (d) to be simultaneous or sequential in any order, provided that it is favourable to the interaction of said compounds X and Y, and
the exposure to a magnetic field of at least a portion of the magnetic substances so as to move them and/or to orientate them, this exposure taking place before the interaction between the compounds X and Y is complete, so that their interaction does not excessively conflict with the modification in the orientation and/or with the movement of the magnetic substances under the effect of the magnetic field.

According to this embodiment, compounds X and Y are capable of reacting by condensation, either in the presence of water (hydrolysis) by reaction of 2 compounds bearing alkoxysilane groups, or by so-called “direct” condensation by reaction of a compound bearing alkoxysilane group(s) and a compound bearing silanol group(s) or by reaction of 2 compounds bearing silanol group(s).

When the condensation is carried out in the presence of water, the latter can in particular be the ambient humidity, the residual water of the skin, of the lips, of the eyelashes and/or of the nails, or water from an external source, for example by prior moistening of the keratinous substance (for example by an atomizer, by natural or artificial tears).

In this manner of reaction by condensation, compounds X and Y, which may be identical or different, can therefore be selected from silicone compounds whose main chain contains at least two alkoxysilane groups and/or at least two silanol (Si—OH) side groups or end groups.

According to one embodiment, compound X and/or compound Y bears at least one polar group, as described above, capable of forming at least one hydrogen bond with keratinous substances.

According to an advantageous embodiment, compounds X and/or Y are selected from the polyorganosiloxanes comprising at least two alkoxysilane groups. By “alkoxysilane group”, we mean a group comprising at least one —Si—OR moiety, R being an alkyl group having from 1 to 6 carbon atoms.

Compounds X and Y are notably selected from the polyorganosiloxanes comprising alkoxysilane end groups, more specifically those which have at least 2 alkoxysilane end groups, preferably trialkoxysilane end groups.

These compounds X and/or Y preferably mostly comprise units of formula:

R⁹ₛSiO_{(4-s)/2}, (IV)

in which the groups R⁹ represent, independently of one another, a radical selected from alkyl groups having from 1 to 6 carbon atoms, phenyl groups, fluoroalkyl groups, and s is equal to 0, 1, 2 or 3. Preferably, groups R⁹ represent, independently of one another, an alkyl group having from 1 to 6 carbon atoms. As alkyl group, we may notably mention methyl, propyl, butyl, hexyl and mixtures thereof, preferably methyl or ethyl. As fluoroalkyl group, we may mention 3,3,3-trifluoropropyl.

According to a particular embodiment, compounds X and Y, which may be identical or different, are polyorganosiloxanes comprising units of formula:

(R⁹₂SiO₂)ᵣ— (V)

in which R⁹ is as described above, preferably R⁹ is a methyl radical, and f is such that the polymer advantageously has a viscosity at 25° C. in the range from 0.5 to 3000 Pa·s, preferably in the range from 5 to 150 Pa·s; for example f can range from 2 to 5000, preferably from 3 to 3000, and more preferably from 5 to 1000.

These polyorganosiloxane compounds X and Y contain at least 2 trialkoxysilane end groups per molecule of polymer, said groups having the following formula

—ZSiR¹ₓ(OR)₃₋ₓ, (VI)

in which:

the radicals R represent, independently, a methyl, ethyl, n-propyl, isopropyl, n-butyl, sec-butyl, isobutyl group, preferably a methyl or ethyl group,

R¹ is a methyl or ethyl group,

x is equal to 0 or 1, preferably x is equal to 0 and

Z is selected from: the divalent hydrocarbon groups that do not have an ethylenic unsaturation and have from 1 to 18 carbon atoms, preferably from 2 to 18 carbon atoms (alkylene groups), the combinations of divalent hydrocarbon radicals and siloxane segments of the following formula (IX):

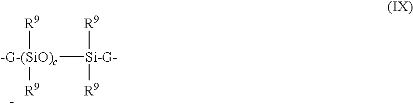

R⁹ being as described above, G is a divalent hydrocarbon radical without an ethylenic unsaturation and having from 1 to 18 carbon atoms, preferably from 2 to 18 carbon atoms and c is an integer in the range from 1 to 6.

Z and G can notably be selected from the alkylene groups such as methylene, ethylene, propylene, butylene, pentylene, hexylene, the arylene groups such as phenylene.

Preferably, Z is an alkylene group, and more preferably ethylene.

These polymers can have on average at least 1.2 trialkoxysilane end groups or terminal chains per molecule, and preferably on average at least 1.5 trialkoxysilane end groups per molecule. These polymers that can have at least 1.2 trialkoxysilane end groups per molecule, some can include other types of end groups such as end groups of formula CH₂═CH—SiR⁹₂— or of formula R⁶₃—Si—, in which R⁹ is as defined previously and each group R⁶ is selected independently from the R⁹ or vinyl groups. As examples of said end groups, we may mention the trimethoxysilane, triethoxysilane, vinyldimethoxysilane and vinylmethyloxyphenylsilane groups.

Such polymers are notably described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the contents of which are incorporated by reference in the present application.

We may mention, as compound X and/or Y, in particular the polyorganosiloxanes selected from the polymers of formula:

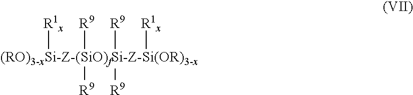

in which R, R¹, R⁹, Z, x and f are as described above.

Compounds X and/or Y can also include a mixture of polymers of formula (VII) above with polymers of the following formula (VIII):

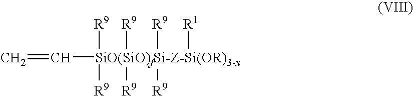

in which R, R¹, R⁹, Z, x, and f are as described above.

When the polyorganosiloxane compound X and/or Y with alkoxysilane group(s) includes said mixture, the various polyorganosiloxanes are present at contents such that the organosilyl terminal chains represent less than 40%, preferably less than 25% in number of terminal chains.

Polyorganosiloxane compounds X and/or Y that are particularly preferred are those of formula (VII) described above. Such compounds X and/or Y are described for example in document WO 01/96450.

As stated above, compounds X and Y can be identical or different.

In particular, compounds X and Y can represent a mixture of polydimethylsiloxanes with methoxysilane groups.

According to a variant, one of the 2 reacting compounds X or Y is of silicone character and the other is of organic character. For example, compound X is selected from organic oligomers or polymers or hybrid organic/silicone oligomers or polymers, said polymers or oligomers comprising at least two alkoxysilane groups, and Y is selected from silicone compounds such as the polyorganosiloxanes described above. In particular, the organic oligomers or polymers are selected from the vinylic, (meth)acrylic oligomers or polymers, polyesters, polyamides, polyurethanes and/or polyureas, polyethers, polyolefins, perfluoropolyethers, dendrimers and hyperbranched organic polymers, and mixtures thereof.

According to one embodiment, compound X of organic character or of hybrid organic/silicone character bears at least one polar group, as described above, capable of forming at least one hydrogen bond with the keratinous substance.

The organic polymers of vinylic or (meth)acrylic character, bearing alkoxysilane side groups, can in particular be obtained by copolymerization of at least one vinylic or (meth)acrylic organic monomer with a (meth)acryloxypropyltrimethoxysilane, a vinyltrimethoxysilane, a vinyltriethoxysilane, an allyltrimethoxysilane etc.

We may mention for example the (meth)acrylic polymers described in the document of KUSABE, M, Pitture e Verniei—European Coating; 12-B, pages 43-49, 2005, and notably the polyacrylates with alkoxysilane groups with the designation MAX from Kaneka or those described in the work by PROBSTER, M, Adhesion-Kleben & Dichten, 2004, 481 (1-2), pages 12-14.

The organic polymers resulting from a polycondensation or a polyaddition, such as polyesters, polyamides, polyurethanes and/or polyureas, polyethers, and bearing alkoxysilane side and/or end groups, can result for example from reaction of an oligomeric prepolymer as described above with one of the following silane reaction partners bearing at least one alkoxysilane group: aminopropyltrimethoxysilane, aminopropyltriethoxysilane, amino ethyl aminopropyl trimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes with alkoxysilane groups are described in the work by KUSABE, M., Pitture e Verniei—European Coating; 12-B, pages 43-49, 2005. As examples of polyurethanes with alkoxysilane end groups, we may mention those described in the document PROBSTER, M., Adhesion-Kleben & Dichten, 2004, 481 (1-2), pages 12-14 or alternatively those described in the document LANDON, S., Pitture e Verniei Vol. 73, No. 11, pages 18-24, 1997 or in the document HUANG, Mowo, Pitture e Verniei Vol. 5, 2000, pages 61-67, and we may notably mention the polyurethanes with alkoxysilane groups from OSI-WITCO-GE.

As polyorganosiloxane compounds X and/or Y, we may mention the resins of type MQ or MT which themselves bear alkoxysilane and/or silanol end groups, for example the poly(isobutylsilsesquioxane) resins functionalized with silanol groups offered under reference SST-S7C41 (three Si—OH groups) by the company Gelest.

2a —Additional Reactive Compound

According to one embodiment, compound X and/or Y can additionally be combined with an additional reactive compound comprising at least two alkoxysilane or silanol groups.

We may mention for example:
one or more organic or mineral particles with alkoxysilane and/or silanol groups on their surface, for example fillers surface-treated with said groups.

2b—Catalyst

The condensation reaction can take place in the presence of a metal-based catalyst which can be present with one or other of the compounds X or Y or can be present on its own. For example, said catalyst can be present in the composition in an encapsulated form if the two compounds X and Y, which it is to cause to interact, are present in this same composition in an unencapsulated form or conversely it can be present there in an unencapsulated form if at least one of compounds X and Y is present in the composition in an encapsulated form. The catalyst for use in this type of reaction is preferably a titanium-based catalyst.

We may notably mention the catalysts based on tetraalkoxytitanium of formula:

Ti(OR²)_{y}(OR³)_{4-y},

in which R² is selected from the tertiary alkyl radicals such as tert-butyl, tert-amyl and 2,4-dimethyl-3-pentyl; R³ represents an alkyl radical having from 1 to 6 carbon atoms, preferably a methyl, ethyl, n-propyl, isopropyl, n-butyl, sec-butyl, hexyl group and y is a number in the range from 3 to 4, preferably from 3.4 to 4.

The catalyst can be present at a content ranging from 0.0001 to 20 wt. % relative to the total weight of the composition containing it.

2c—Diluent

The compositions that can be used, comprising X and/or Y, can additionally include a volatile silicone oil (or diluent) for lowering the viscosity of the composition. Said oil can be selected from the short-chain linear silicones such as hexamethyldisiloxane, octamethyltrisiloxane, cyclic silicones such as octamethylcyclotetrasiloxane, decamethylcyclopentasiloxane and mixtures thereof.

This silicone oil can represent from 5 to 95 wt. %, preferably from 10 to 80 wt. % relative to the weight of each composition.

As an example of a combination of compounds X and Y bearing alkoxysilane groups and reacting by condensation, we may mention the combination of the following mixtures A′ and B′ produced by the company Dow Corning:

| | | | |
|---|---|---|---|
| Ingredient (INCI name) | CAS No. | Contents (%) | Function |

Mixture A′:

| | | | |
|---|---|---|---|
| Bis-Trimethoxysiloxyethyl | PMN87176 | 25-45 | Polymer |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| | | | |
|---|---|---|---|
| Disiloxane | 107-46-0 | 80-99 | Solvent |
| Tetra T Butyl Titanate | — | 1-20 | Catalyst |

It should be noted that compounds X and Y, identical, are combined in mixture A′ (cf (1))

3/ Crosslinking in the Presence of Peroxide:

According to one embodiment, the invention relates to a method for making up a support comprising at least:
the application to said support of at least (a) magnetic substances exhibiting a non-zero magnetic susceptibility, (b) one or more compounds X, (c) one of more compounds Y, with at least one of the compounds X and Y being a silicone compound and said compounds X and Y being capable of reacting together by a crosslinking reaction in the presence of a peroxide, when they are brought into contact with one another, and (d) at least one peroxide, it being possible for the applications (a), (b), (c) and (d) to be simultaneous or sequential in any order, provided that it is favourable to the interaction of said compounds X and Y, and
the exposure to a magnetic field of at least a portion of the magnetic substances so as to move them and/or to orientate them, this exposure taking place before the interaction between the compounds X and Y is complete, so that their interaction does not excessively conflict with the modification in the orientation and/or with the movement of the magnetic substances under the effect of the magnetic field.

This reaction is preferably effected by heating to a temperature greater than or equal to 50° C., preferably greater than or equal to 80° C., and up to 120° C.

Compounds X and Y, which may be identical or different, have in this case at least two —CH₃ side groups and/or at least two side chains bearing a —CH₃ group.

Compounds X and Y are preferably silicone compounds and can be selected for example from the non-volatile linear polydimethylsiloxanes of high molecular weight, having a degree of polymerization above 6 and with at least two —CH₃ side groups attached to the silicon atom and/or at least two side chains bearing a —CH₃ group. We may mention for example the polymers described in the Catalogue “Reactive Silicones” of the company Gelest Inc., Edition 2004, page 6, and notably the copolymers (also called gums) of vinylmethylsiloxane-dimethylsiloxane of molecular weight in the range from 500 000 to 900 000 and notably with viscosity above 2 000 000 cSt.

As peroxides that can be used in the invention, we may mention benzoyl peroxide, 2,4-dichlorobenzoyl peroxide and mixtures thereof.

According to one embodiment, the hydrosilylation reaction in the presence of a catalyst, or the condensation reaction, or alternatively the crosslinking reaction in the presence of a peroxide, between compounds X and Y is accelerated by supply of heat, for example by raising the temperature of the system between 25° C. and 180° C.

In general, regardless of the type of reaction by which compounds X and Y react with one another, the molar percentage of X relative to the total of compounds X and Y, i.e. the ratio X/(X+Y)×100, can vary from 5 to 95%, preferably from 10 to 90%, and more preferably from 20 to 80%.

Similarly, the molar percentage of Y relative to the total of compounds X and Y, i.e. the ratio Y/(X+Y)×100, can vary from 5 to 95%, preferably from 10 to 90%, and more preferably from 20 to 80%.

Compound X can have a weight-average molecular weight (Mw) in the range from 150 to 1 000 000, preferably from 200 to 800 000, more preferably from 200 to 250 000.

Compound Y can have a weight-average molecular weight (Mw) in the range from 200 to 1 000 000, preferably from 300 to 800 000, more preferably from 500 to 250 000.

Compound X can represent from 0.1 to 95 wt. % relative to the total weight of the composition containing it, preferably from 1 to 90%, and more preferably from 5 to 80%.

Compound Y can represent from 0.1 to 95 wt. % relative to the total weight of the composition containing it, preferably from 1 to 90%, and more preferably from 5 to 80%.

The ratio of compound X to compound Y can be varied so as to adjust the reaction rate and therefore the rate of formation of the film or alternatively so as to adapt the properties of the film formed (for example its adhesive properties) according to the intended application.

In particular, compounds X and Y can be present at a molar ratio X/Y in the range from 0.05 to 20 and preferably from 0.1 to 10.

Compounds X and Y can advantageously be combined with at least one filler. Thus, the kit according to the invention can for example include, in at least one of the compositions, a filler selected from silica or surface-treated silica.

As pointed out previously, according to one embodiment of the invention, compounds X and Y can be used in the form of a single composition which then contains at least one of them or, if applicable, the catalyst or the peroxide if necessary for their interaction, in an encapsulated form.

Within the scope of the present invention, consideration is given more particularly to the encapsulated forms of the core/shell type, also called microcapsules or nanocapsules, in which the shell is of polymeric character and the core contains compound X, compound Y, one of its compounds X and Y possibly being encapsulated with the catalyst or the peroxide if necessary for the interaction of the two compounds. In the case when this catalyst is not encapsulated with one or other of the compounds X or Y, it is present in the cosmetic composition containing the encapsulated forms.

Numerous techniques are currently available for making microcapsules or nanocapsules of this type.

However, according to a preferred embodiment, the encapsulated forms considered according to the invention are nanocapsules and are obtained by a technique called solvent nanoprecipitation, notably described in documents EP 274 961 and EP 1 552 820.

More particularly, the shell of the nanocapsules of compound X or Y, employed according to the invention, is of polymeric character, not crosslinked, not water-soluble and not soluble in the capsule core.

In general, all the polymers, of natural or synthetic origin, soluble in a solvent that is not miscible with water, and notably those having a melting point below the boiling point of water at atmospheric pressure (100° C.), may be suitable.

These polymers can be biodegradable, for example polyesters, or non-biodegradable.

By way of illustration of polymers that are suitable for the invention, we may notably mention:
C₂-C₁₂ alkyl cyanoacrylate polymers
polymers formed by poly-L-lactides, poly-DL-lactides, polyglycolides and the corresponding copolymers,
polycaprolactones,
polymers of 3-hydroxybutyric acid,
copolymers of vinyl chloride and vinyl acetate,
copolymers of methacrylic acid and methacrylic ester, notably of methacrylic acid and of methacrylate,
polyvinyl acetophthalate,
cellulose acetophthalate,
polyvinylpyrrolidone-vinyl acetate copolymer,
polyethylenevinyl acetates,
polyacrylonitriles,
polyacrylamides,
polyethylene glycols,
poly-(C₁ to C₄ hydroxyalkyl methacrylate)
esters of cellulose and C₁-C₄ carboxylic acid,
polystyrene and copolymers of styrene and maleic anhydride, copolymers of styrene and acrylic acid, styrene ethylene/butylene-styrene block terpolymers, styrene-ethylene/propylene-styrene block terpolymers,
styrene alkyl-alcohol oligomers,
terpolymers of ethylene, vinyl acetate and maleic anhydride,
polyamides,
polyethylenes,
polypropylenes,
organopolysiloxanes including polydimethylsiloxanes,
poly(alkylene adipate),
polyol polyesters,
polysilsesquioxane silicone polymers,
dendritic polyesters with a hydroxyl terminal function,
polymers that are water-dispersible but are nevertheless soluble in solvents that are not miscible with water, for example: polyesters, poly(ester amides), polyurethanes and vinyl copolymers bearing carboxylic and/or sulphonic acid functions and in particular those described in document FR 2 787 729,
block copolymers insoluble in water at room temperature and solid at room temperature, having at least one block of one of the aforementioned polymers, and
mixtures thereof.

These polymers or copolymers can have a weight-average molecular weight between 1000 and 500 000 and in particular between 1500 and 100 000.

The following are quite particularly suitable for the invention: poly(alkylene adipate), organopolysiloxanes, polycaprolactones, cellulose acetophthalate, cellulose acetobutyrate, cellulose esters, polystyrene and its derivatives and notably polycaprolactones.

Of course, a person skilled in the art is able, on the basis of his knowledge, to adjust the molecular weight of the polymer selected with respect to its concentration in the solvent so as have a mixture viscosity compatible with satisfactory emulsification.

With regard to the lipophilic core, it can contain at least one oil, in addition to compound X or compound Y. Said oil can be selected from the oils described hereunder for the oily phase. The oil is preferably a silicone oil.

According to a variant of the invention, the encapsulated forms of compound X or compound Y can be coated with a lamellar phase.

Regarding the operating procedure for production of nanocapsules suitable for the invention, a person skilled in the art can notably refer to the teaching in document EP 1 552 820 cited previously. The choice of the necessary surfactants as well as the carrying out of the method requires the knowledge of a person skilled in the art.

Other Components

The compositions for the implementation of the invention, in particular those comprising magnetic substances, comprise at least one physiologically acceptable medium.

The term “physiologically acceptable medium”, synonymous with “cosmetically acceptable medium”, denotes a nontoxic medium capable of being applied to the skin, superficial body growths or lips of human beings.

The physiologically acceptable medium is generally suited to the nature of the support on which the composition has to be applied and also to the form under which the composition is packaged.

The compositions can comprise other ingredients than those mentioned above, in particular at least one solvent, one fatty phase, one film-forming polymer and/or one dermatological or cosmetic active principle, in particular according to the formulation form, as described in detail below.

Solvents

The compositions can comprise at least one aqueous or organic solvent, in particular a volatile organic solvent.

The compositions can comprise a volatile solvent, in particular a volatile organic solvent.

Within the meaning of the present invention, the term “volatile solvent” is understood to mean a solvent which is liquid at ambient temperature and which has in particular a non-zero vapour pressure, at ambient temperature and atmospheric pressure, in particular which has a vapour pressure ranging from 0.13 Pa to 40 000 Pa (10⁻³ to 300 mm Hg), preferably ranging from 1.3 Pa to 13 000 Pa (0.01 to 100 mm Hg) and preferentially ranging from 1.3 Pa to 1300 Pa (0.01 to 10 mm Hg).

When the compositions comprise one or more organic solvents, these solvents can be present in a content ranging from 0.1% to 99%, with respect to the total weight of the composition concerned.

Generally, the amount of solvent(s), in particular organic solvent(s), will depend on the nature of the support on which the compositions are intended to be applied.

The compositions can comprise at least one volatile solvent composed of a volatile oil.

The oil can be a silicone oil or a hydrocarbon oil or can comprise a mixture of such oils.

Within the meaning of the present invention, the term “silicone oil” is understood to mean an oil comprising at least one silicon atom and in particular at least one Si—O group.

The term hydrocarbon oil is understood to mean an oil mainly comprising hydrogen and carbon atoms and optionally oxygen, nitrogen, sulphur and/or phosphorus atoms.

The volatile hydrocarbon oils can be chosen from hydrocarbon oils having from 8 to 16 carbon atoms and in particular branched C8-C16 alkanes (also known as isoparaffins), such as isododecane (also known as 2,2,4,4,6-pentamethylheptane), isodecane, isohexadecane and, for example, the oils sold under the Isopar® or Permethyl® trade names.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 10 silicon atoms, in particular from 2 to 7 silicon atoms, these silicones optionally comprising alkyl or alkoxy groups having from 1 to 10 carbon atoms. Mention may in particular be made, as volatile silicone oil which can be used in the invention, of dimethicones with a viscosity of 5 and 6 cSt, octamethylcyclotetrasiloxane, decamethylcyclopentasiloxane, dodecamethylcyclo-hexasiloxane, heptamethylhexyltrisiloxane, heptamethyloctyltrisiloxane, hexamethyl-disiloxane, octamethyltrisiloxane, decamethyltetrasiloxane, dodecamethylpentasiloxane, and their mixtures.

Mention may also be made of volatile linear alkyltrisiloxane oils of general formula (I):

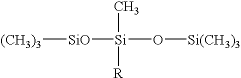

where R represents an alkyl group comprising from 2 to 4 carbon atoms, one or more hydrogen atoms of which can be substituted by a fluorine or chlorine atom.

Mention may be made, among the oils of general formula (I), of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,

corresponding to the oils of formula (I) for which R is respectively a butyl group, a propyl group or an ethyl group.

Use may also be made of volatile fluorinated oils, such as nonafluoromethoxybutane or perfluoromethylcyclopentane, and their mixtures.

The compositions can comprise, for example, between 0.01% and 95% by weight of volatile oil, with respect to the total weight of the composition, better still between 1% and 75% by weight.

The compositions can comprise at least one organic solvent chosen from the following list:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
glcyols which are liquid at ambient temperature, such as ethylene glycol, propylene glycol, pentylene glycol or glycerol;
propylene glycol ethers which are liquid at ambient temperature, such as propylene glycol monomethyl ether, propylene glycol monomethyl ether acetate or dipropylene glycol mono(n-butyl)ether;
short-chain esters (having from 3 to 8 carbon atoms in total), such as ethyl acetate, methyl acetate, propyl acetate, n-butyl acetate or isopentyl acetate;
alkanes which are liquid at ambient temperature, such as decane, heptane, dodecane or cyclohexane.

The compositions can also comprise water or a mixture of water and of hydrophilic organic solvents commonly used in cosmetics, such as alcohols, in particular linear or branched lower monoalcohols having from 2 to 5 carbon atoms, such as ethanol, isopropanol or n-propanol, or polyols, such as glycerol, diglycerol, propylene glycol, sorbitol, pentylene glycol or polyethylene glycols. The first composition can additionally comprise hydrophilic C₂ ethers and C₂-C₄ aldehydes. Water or the mixture of water and of hydrophilic organic solvents can be present in the first and/or the second composition in a content ranging, for example, from 0% to 90% by weight, in particular from 0.1% to 90% by weight and preferably from 0% to 60% by weight, in particular from 0.1% to 60% by weight, with respect to the total weight of the composition.

Fatty Phase

The compositions, for example when they are intended to be applied to the lips or the eyelashes, can comprise a fatty phase and in particular at least one fatty substance which is liquid at ambient temperature (25° C.) and under atmospheric pressure (760 mm Hg) and/or one fatty substance which is solid at ambient temperature, such as waxes, pasty fatty substances, gums and their mixtures. The fatty phase can additionally comprise lipophilic organic solvents.

The compositions can exhibit, for example, a continuous fatty phase which can comprise less than 5% of water, in particular less than 1% of water, with respect to its total weight, and in particular can be in the anhydrous form.

As fatty substances which are liquid at ambient temperature, also known as “oils”, mention may be made of: vegetable hydrocarbon oils, such as liquid triglycerides of fatty acids having from 4 to 10 carbon atoms, such as triglycerides of heptanoic or octanoic acids, or also sunflower, maize, soybean, grape seed, sesame, apricot, macadamia, castor or avocado oils, triglycerides of caprylic/capric acids, jojoba oil, shea butter oil, lanolin oil or acetylated lanolin oil; linear or branched hydrocarbons of mineral or synthetic origin, such as liquid paraffins and their derivatives, liquid petrolatum, polydecenes or hydrogenated polyisobutene, such as parleam; synthetic esters and ethers, in particular of fatty acids, such as, for example, Purcellin oil, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate or triisocetyl citrate; heptanoates, octanoates or decanoates of fatty alcohols; isononyl isononanoate, isopropyl lanolate, tridecyl trimellitate, diisostearyl malate; polyol esters, such as propylene glycol dioctanoate, neopentyl glycol diheptanoate or diethylene glycol diisononanoate; pentaerythritol esters; fatty alcohols having from 12 to 26 carbon atoms, such as octyldodecanol, 2-butyloctanol, 2-hexyldecanol, 2-undecylpentadecanol or oleyl alcohol; partially hydrocarbon-comprising and/or silicone-comprising fluorinated oils; silicone oils, such as volatile or nonvolatile and linear or cyclic polymethylsiloxanes (PDMSs) which are liquid or pasty at ambient temperature, such as cyclomethicones, dimethicones, optionally comprising a phenyl group, such as phenyl trimethicones, phenyltrimethylsiloxydiphenylsiloxanes, diphenylmethyldimethyl-trisiloxanes, diphenyl dimethicones, phenyl dimethicones or polymethylphenylsiloxanes; their mixtures. The oils can be present in a content ranging from 0.01 to 90% by weight and better still from 0.1 to 85% by weight, with respect to the total weight of the composition.

The presence of an oily phase can confer gloss and exhibit, for example, a refractive index of between 1.47 and 1.51, better still between 1.48 and 1.50. The refractive index is measured at ambient temperature (25° C.) using a refractometer.

The compositions can comprise at least one agent for structuring the liquid fatty phase (formed by the volatile or nonvolatile oils and/or organic solvents described above) chosen from waxes, semicrystalline polymers, lipophilic gelling agents and their mixtures.

The pasty fatty substances are generally hydrocarbon compounds with a melting point of between 25 and 60° C., preferably between 30 and 45° C., and/or a hardness of between 0.001 and 0.5 MPa, preferably between 0.005 and 0.4 MPa, such as lanolins and their derivatives.

The waxes can be solid at ambient temperature (25° C.), with a reversible solid/liquid change in state, having a melting point of greater than 30° C. which can range up to 200° C., having a hardness of greater than 0.5 MPa and exhibiting, in the solid state, an anisotropic crystalline arrangement. In particular, the waxes can exhibit a melting point of greater than 25° C. and better still of greater than 45° C. The waxes can be hydrocarbon, fluorinated and/or silicone waxes and can be of vegetable, mineral, animal and/or synthetic origin. Mention may be made, as waxes which can be used, of beeswax, carnauba wax, candelilla wax, paraffin wax, microcrystalline waxes, ceresin or ozokerite; synthetic waxes, such as polyethylene or Fischer Tropsch waxes, silicone waxes, such as alkyl or alkoxy dimethicone having from 16 to 45 carbon atoms. The composition can comprise from 0 to 50% by weight of waxes, with respect to the total weight of the composition, indeed even from 1 to 30% by weight.

The gums which can be used are generally polydimethylsiloxanes (PDMSs) of high molecular weight or cellulose gums or polysaccharides.

Film-Forming Polymers

The compositions can also comprise, for example, a film-forming polymer, in particular in the case of a mascara, of a nail polish or of a foundation. The term “film-forming polymer” denotes a polymer capable of forming, by itself alone or in the presence of an additional agent which is able to form a film, a continuous film which adheres to a support, in particular to keratinous substances.

Mention may be made, among the film-forming polymers which can be used in the compositions according to the invention, inter alia, of synthetic polymers of radical or polycondensate type, polymers of natural origin, such as nitrocellulose or cellulose esters, and their mixtures.

The film-forming polymers of radical type can in particular be vinyl polymers or copolymers, in particular acrylic polymers.

The film-forming vinyl polymers can result from the polymerization of monomers possessing ethylenic unsaturation having at least one acid group and/or of the esters of these acid monomers and/or of the amides of these acid monomers, such as α,β-ethylenic unsaturated carboxylic acids, for example acrylic acid, methacrylic acid, crotonic acid, maleic acid or itaconic acid.

The film-forming vinyl polymers can also result from the homopolymerization or from the copolymerization of monomers chosen from vinyl esters, such as vinyl acetate, vinyl neodecanoate, vinyl pivalate, vinyl benzoate and vinyl t-butylbenzoate, and styrene monomers, such as styrene and α-methylstyrene.

Mention may be made, among the film-forming polycondensates, of polyurethanes, polyesters, polyesteramides, polyamides and polyureas, this list not being limiting.

The polymers of natural origin, which are optionally modified, can be chosen from shellac resin, gum sandarac, dammars, elemis, copals, cellulose polymers, such as nitrocellulose, ethylcellulose or cellulose esters chosen, for example, from cellulose acetate, cellulose acetate butyrate or cellulose acetate propionate, and their mixtures.

The film-forming polymer can be present in the form of solid particles in aqueous or oily dispersion, generally known under the name of latex or pseudo latex. The film-forming polymer can comprise one or more stable dispersions of generally spherical polymeric particles of one or more polymers in a physiologically acceptable liquid fatty phase. These dispersions are generally referred to as NAD (non-aqueous dispersion) of polymer, in contrast to latexes, which are aqueous dispersions of polymer. These dispersions can in particular be provided in the form of nanoparticles of polymers in stable dispersion in said fatty phase. The nanoparticles preferably have a size of between 5 and 600 nm. The techniques for preparing these dispersions are well known to a person skilled in the art.

Use may be made, as aqueous dispersion of film-forming polymer, of the acrylic dispersions sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical or Daitosol 5000 AD® by Daito Kasei Kogyo; or also the aqueous polyurethane dispersions sold under the names Neorez R-981® or Neorez R-974® by Avecia-Neoresins, Avalure UR-405®, Avalure UR-410®, Avalure UR-425®, Avalure UR-450®, Sancure 875®, Sancure 861®. Sancure 878® and Sancure 2060® by Goodrich, Impranil 85® by Bayer or Aquamere H-1511® by Hydromer; or the sulphopolyesters sold under the trade name Eastman AQ by Eastman Chemical Products.

Film-Forming Block Polymer

The compositions can comprise at least one film-forming polymer which is a film-forming linear ethylenic block polymer. This polymer preferably comprises at least one first block and at least one second block having different glass transition temperatures (Tg), said first and second blocks being connected to one another via an intermediate block comprising at least one constituent monomer of the first block and at least one constituent monomer of the second block.

Advantageously, the first and second blocks of the block polymer are incompatible with one another.

Such polymers are described, for example, in the documents EP 1411069 or WO 04/028488, which are incorporated by reference.

The compositions can also comprise an additional agent which is able to form a film which promotes the formation of a film with the film-forming polymer.

The compositions can comprise at least one magnetic or nonmagnetic filler.

Fillers

The term “filler” denotes particles of any shape which are insoluble in the medium of the composition concerned, whatever the temperature at which the composition is manufactured. A filler can be used in particular to modify the rheology or the texture of the composition. The nature and the amount of the particles can depend on the mechanical properties and textures desired.

Mention may be made, as example of fillers, inter alia, of talc, mica, silica, kaolin, sericite, powders derived from polyamide, from polyolefin, for example from polyethylene, from polytetrafluoroethylene, from polymethyl methacrylate or from polyurethane, starch powders and silicone resin beads.

The fillers may be intended to create, inter alia, a soft focus effect, in particular in the case of a foundation, in order to conceal imperfections of the skin.

Active Principles

These compositions can comprise at least one cosmetic or dermatological active principle. Mention may be made, as cosmetic, dermatological, hygiene or pharmaceutical active principles which can be used in the compositions of the invention, of moisturizing agents (polyol, such as glycerol), vitamins (C, A, E, F, B or PP), essential fatty acids, essential oils, ceramides, sphingolipids, fat-soluble sunscreens or sunscreens in the form of nanoparticles, specific active principles for treatment of the skin (protective agents, antibacterials, antiwrinkle active agents, and the like) or self-tanning agents. These active principles can be used, for example, at concentrations of 0 to 20% and in particular of 0.001 to 15%, with respect to the total weight of the composition.

The compositions can also comprise ingredients commonly used in cosmetics, such as, for example, thickening agents, surfactants, trace elements, moisturizing agents, softening agents, sequestering agents, fragrances, basifying or acidifying agents, preservatives, antioxidants, UV screening agents, colorants or their mixtures.

The compositions can comprise, depending on the type of application envisaged, the constituents conventionally used in the fields under consideration, which are present in an amount appropriate to the formulation form desired.

Formulation Forms

The compositions can be provided in various forms depending on their destination.

The compositions can thus be provided in any formulation form normally used for topical application and in particular in the anhydrous form or in the form of an oily or aqueous solution, of an oily or aqueous gel, of an oil-in-water, water-in-oil, wax-in-water or water-in-wax emulsion, of a multiple emulsion or of a dispersion of oil in water by virtue of vesicles situated at the oil/water interface.

Magnetic Devices

The magnetic field is created using a magnetic device.

This magnetic device can comprise a permanent magnet or an electromagnet, for example fed by at least one battery or storage battery. In the latter case, the magnetic device can comprise a switch which makes it possible to selectively feed the electromagnet with electricity.

The magnetic device can be arranged in order to create a magnetic field, the orientation of which varies over time. When the magnetic device comprises a magnet, the device can, for example, comprise a motor which makes it possible to drive the magnet in rotation. In an alternative form, the magnetic device can comprise several solenoids arranged so as to generate, when fed sequentially with electricity, a rotating magnetic field.

A rotating magnetic field can make it possible, for example, to obtain a pattern exhibiting rotational symmetry, for example a pattern which gives the impression of a sphere in relief.

The electromagnetic or electromagnets can be continuously or intermittently fed, as chosen by the user. In particular, the magnetic device can be arranged so that the electromagnet or electromagnets may not be fed as long as the magnetic device is not positioned correctly close to the support coated with the first composition.

The magnetic field is, for example, at least 50 mT, indeed even at least 66 mT, better still at least 0.2 T, indeed even at least 1 T (10 000 Gauss).

So as to make it easier to apply the magnetic field, the magnetic device can comprise a means which makes it possible to position it relative to the support on which the composition has been deposited. This can make it possible, for example, to avoid the magnetic device from accidentally coming into contact with the composition and/or to centre the pattern produced on the region concerned.

In an exemplary embodiment of the invention, the magnetic device is integral with an applicator used for the application of the cosmetic composition. This can make it possible to reduce the number of objects handled by the user and to make it easier to put on the makeup.

In another exemplary embodiment of the invention, the magnetic device contained in a kit according to the present invention comprises a magnet, which may be fitted at a first end of a rod, the second end of which is connected to a grasping means of an applicator used for the application of the cosmetic composition.

The magnetic field can also be exerted by means of a magnetic structure, in particular a flexible magnetic structure, comprising an alternation of N and S poles. Such a structure can make it possible, for example, to produce repetitive patterns on the first composition, for example stripes.

Kits for the Implementation of the Method

A further subject-matter of the invention is, according to another of its aspects, a kit for the implementation of a method as defined above, comprising at least:
one magnetic device which makes it possible to generate a magnetic field, and
at least two different compositions packaged separately, the kit comprising at least magnetic substances exhibiting a nonzero magnetic susceptibility, at least one compound X, at least one compound Y and optionally at least one catalyst or one peroxide, at least one of the compounds X or Y being a silicone compound, with the compounds X, Y and the optional catalyst or the optional peroxide, when they are present, not being present simultaneously in the same composition, said compounds X and Y being capable of reacting together by a hydrosilylation reaction in the presence of a catalyst or by a condensation reaction or by a crosslinking reaction in the presence of a peroxide, when they are brought into contact with one another,
   the magnetic device being capable of creating a magnetic field which is able, when a support covered with a deposited layer comprising at least magnetic substances is introduced into the magnetic field, to modify the orientation and/or the position of the magnetic substances inside the deposited layer.

The kit can in particular be a cosmetic kit, comprising at least:
a magnetic device which makes it possible to generate a magnetic field,
a first composition comprising, in a physiologically acceptable medium, at least one compound X, and
a second composition comprising, in a physiologically acceptable medium, at least one compound Y,
   with at least one of the first and second compositions additionally comprising at least magnetic substances exhibiting a nonzero magnetic susceptibility, and at least one of said first and second compositions containing, additionally, where appropriate, at least one catalyst or a peroxide.

Preferably, the first composition comprising the compound X and the second composition comprising the compound Y are packaged in separate packagings.

For example, each composition can be packaged separately in the same packaging article, for example in a two-compartment pen, the base composition being delivered via one end of the pen and the top composition being delivered via the other end of the pen, each end being, in particular, hermetically closed by a cap. Each composition can also be packaged in a compartment within the same packaging article, the mixing of the two compositions being carried out, for example, at the end or ends of the packaging article during the delivery of each composition.

Alternatively, each of the first and second compositions can be packaged in different packaging articles.

The first and second compositions are different from one another.

For example, the first composition is advantageously devoid of compound Y and the second composition is advantageously devoid of compound X. This is because, in view of their high reactivity with regard to one another, the compounds X and Y are not present simultaneously in a first and/or second composition forming a kit according to the invention when their interaction is not conditioned by the presence of a catalyst or of a peroxide.

On the other hand, at the time of application or immediately before, the compounds X and Y are mixed and present in the same composition, resulting from the mixing of the first and second compositions forming the kit according to the invention.

According to another alternative embodiment, the composition applied comprises at least one of the compounds X and Y in an encapsulated form.

Thus, the present invention is targeted, according to yet another of its aspects, at a cosmetic kit comprising, in a physiologically acceptable medium, at least magnetic substances exhibiting a nonzero magnetic susceptibility, one or more compounds X, one or more compounds Y, with at least one of the compounds X and Y being a silicone compound and said compounds X and Y being capable of reacting together via a hydrosilylation reaction in the presence of a catalyst or via a condensation reaction or via a crosslinking reaction in the presence of a peroxide, when they are brought into contact with one another, and, where appropriate, at least one catalyst or one peroxide, with at least one of the compounds X and Y being present in an encapsulated form.

One of the compositions of the kit according to the invention can thus comprise the magnetic substances and at least one compound X, respectively one compound Y, and the other composition can comprise at least one compound Y, respectively one compound X, with at least one of the compositions containing, additionally, where appropriate, at least one catalyst or one peroxide.

The catalyst or catalysts and/or peroxide or peroxides can be present in either of the compositions depending on the compatibility with the remainder of the ingredients, indeed even can be packaged separately from the first and second compositions.

The two above compositions can be packaged in different independent containers or, in an alternative form, in the same packaging device comprising two compartments which can be brought into communication, in an extemporaneous way.

If appropriate, the catalyst or catalysts and/or peroxide or peroxides can be present in a compartment of a packaging device comprising one of the two abovementioned compositions, it being possible for this compartment to be brought into communication, in an extemporaneous way, with that comprising the other composition.

In one example, each of the above compositions is present in a respective compartment of a packaging device in the form, for example, of a pen, the compartments being closed by a closing means which can pass from a state of closure to a state of nonclosure in response to an action of the user on the packaging device, for example a rotation or the movement of a part of the device.

The magnetic device can be packaged with the two above compositions at least, for example in a single packaging. In an alternative form, the magnetic device can be provided to the consumer separately. If appropriate, the magnetic device can be integral with one of the packaging devices of one of the compositions, for example being integral with an applicator or with a container comprising this composition.

In the case where the two above compositions are applied successively, the application of the second composition can be carried out, for example, while seeking to disturb as little as possible the underlying layer of the other composition, for example using a flocked applicator or by spraying, it being possible for the spraying to take place, for example, using a piezoelectric, electrostatic spraying device or an airbrush, which can, if appropriate, form part of the kit.

When the two compositions are mixed in an extemporaneous way, the kit advantageously comprises a mixer which makes it possible to homogenize the mixture. It can be, for example, a rotating means or ball.

The compositions can be packaged in an amount corresponding to a single use after mixing the compositions. In an alternative form, the compositions can be packaged each in an amount suitable for several successive applications.

If appropriate, the two compositions can be extracted from two respective containers or compartments and can pass through a mixer before application to the keratinous substances.

The above compositions can be packaged in air-filled or airless containers, depending on the preservation desired.

The magnetic device can be arranged in order to generate a magnetic field which is sufficiently strong to be able to modify the orientation and/or the position of the magnetic substances within the composition comprising the magnetic substances after application to a support composed, for example, of keratinous substances, such as the skin, mucous membranes or superficial body growths, so as to change the appearance thereof.

The magnetic field is, for example, exerted shortly after the deposition, so as to change the appearance of the composition comprising the magnetic substances before the composition dries, for example when the composition comprises a volatile solvent.

The compositions of the kit can, for example, be intended to form a nail polish, a foundation or a product to be applied to the lips, such as, for example, a lipstick, or also a product to be applied to keratinous fibres, for example to the eyelashes.

The kit can comprise a case housing the first and second cosmetic compositions and the magnetic device. In this case, the case can comprise, for example, a plurality of magnets of different shapes in order to produce different patterns.

The kit can additionally comprise an additional cosmetic composition to be applied to the abovementioned compositions or to the treated support.

### EXAMPLES PROPOSED

In the examples of compositions described hereunder, the combination of the following mixtures A and B produced by the company Dow Corning is used as combination of compounds X and Y:

| | | | |
|---|---|---|---|
| Ingredient (INCI name) | CAS No. | Contents (%) | Function |

MIXTURE A:

| | | | |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| | | | |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

### Example 1

### Lipstick

First composition (proportions by weight)

| | |
|---|---|
| Mixture A | 50.00% |
| DC Red 7 | 7.27% |
| Phenyl trimethicone 1000 cSt | 14.90% |
| Phenyl trimethicone 20 cSt | 27.83% |

Second composition (proportions by weight)

| | |
|---|---|
| Mixture B | 50.00% |
| Magnetic pigment (Colorona Blackstar Red ® from | 3.00% |
| Engelhard) | |
| Phenyl trimethicone 1000 cSt | 23.10% |
| Phenyl trimethicone 20 cST | 23.90% |

Procedure

First of all, a homogenate of pigment material is produced in phenyl trimethicone 20 cSt.

The various constituents of each composition are then mixed, so as to form the first and second compositions, which are subsequently placed under magnetic stirring.

Application

The first composition is applied so as to create a continuous coloured background.

Subsequently, the second composition is applied and then the deposited layer formed by the first and second compositions is exposed to a magnetic field.

A variation in the appearance under the effect of the magnetic field is observed.

### Example 2

### Anhydrous Foundation

First composition (% by weight)

| | |
|---|---|
| Mixture A | 30% |
| PMMA (Covabead LH85) | 20% |
| Isononyl isononanoate | 50% |

Second composition (% by weight)
Phase A

| | |
|---|---|
| Mixture B | 30% |

Phase B

| | |
|---|---|
| Magnetic pigment | 3% |
| Titanium dioxide coated with isopropyl titanium | 9.5% |
| triisostearate, sold under the reference BTD-401 | |
| by Kobo | |
| Isononyl isononanoate | 7.5% |

Phase C

| | |
|---|---|
| Isononyl isononanoate | 50% |

Procedure

Phase B is prepared by milling the pigments 3 times on a triple roll mill.

Phases A, B and C are subsequently combined together and mixed using a magnetic stirrerd.

The invention is not limited to the examples described, which are given solely by way of illustration.

The expression “comprising a” should be understood as synonymous with “comprising at least one”, unless otherwise specified.

1. Method for making up a support, comprising at least:
the application to said support of at least (a) magnetic substances exhibiting a non-zero magnetic susceptibility, (b) one or more compounds X, (c) one of more compounds Y, with at least one of the compounds X and Y being a silicone compound and said compounds X and Y being capable of reacting together by a hydrosilylation reaction in the presence of a catalyst, when they are brought into contact with one another, and (d) at least one catalyst, it being possible for the applications (a), (b), (c) and (d) to be simultaneous or sequential in any order, provided that it is favourable to the interaction of said compounds X and Y, and
the exposure to a magnetic field of at least a portion of the magnetic substances so as to move them and/or to orientate them, this exposure taking place before the interaction between the compounds X and Y is complete, so that their interaction does not excessively conflict with the modification in the orientation and/or with the movement of the magnetic substances under the effect of the magnetic field.

2. Method according to claim 1, comprising at least:
the application to the support of at least one first cosmetic composition comprising, in a physiologically acceptable medium, said magnetic substances and at least one compound X, respectively at least one compound Y,
the exposure of the magnetic substances to the magnetic field, so as to modify their orientation and/or to move them, and
the application to the first cosmetic composition of at least one second cosmetic composition comprising, in a physiologically acceptable medium, at least one compound Y, respectively at least one compound X, with at least one of said first and second compositions comprising, additionally, at least one catalyst,
the exposure of the magnetic substances to the magnetic field, so as to modify their orientation and/or to move them, taking place prior or subsequent to the application of the second composition.

3. Method according to claim 1, comprising at least:
the application to the support of at least one first cosmetic composition comprising, in a physiologically acceptable medium, said magnetic substances, and
the application to the first cosmetic composition of at least one second cosmetic composition comprising, in a physiologically acceptable medium, at least one compound X at least one compound Y, and at least one catalyst,
the exposure of the magnetic substances to the magnetic field, so as to modify their orientation and/or to move them, taking place prior or subsequent to the application of the second composition but before the compounds X and Y have completed their interaction.

4. Method according to claim 3, the second composition resulting from the extemporaneous mixing of at least two separate products, one comprising at least the compound or compounds X and the other comprising at least the compound or compounds Y, with at least one of the first and second compositions comprising, additionally, at least one catalyst.

5. Method according to claim 1, comprising at least:
the application to said support of at least one first cosmetic composition comprising, in a physiologically acceptable medium, at least one compound X, respectively at least one compound Y, followed by
the application to the first composition of at least one second composition comprising, in a physiologically acceptable medium, at least one compound Y, respectively at least one compound X, and said magnetic substances, with at least one of said first and second compositions comprising, additionally, at least one catalyst.

6. Method according claim 5, the exposure of the magnetic substances to the magnetic field, so as to modify their orientation and/or to move them, taking place subsequent to the application of the second composition but before the compounds X and Y have completed their interaction.

7. Method according to claim 1, comprising at least:
the application to said support of at least one first cosmetic composition comprising, in a physiologically acceptable medium, said magnetic substances, followed by
the application to the first composition of at least one second cosmetic composition comprising, in a physiologically acceptable medium, at least one compound X, respectively at least one compound Y, and
the application to the second cosmetic composition of at least one third cosmetic composition comprising, in a physiologically acceptable medium, at least one compound Y, respectively at least one compound X, with at least one of the compositions comprising, additionally, at least one catalyst.

8. Method according to claim 7, the exposure of the magnetic substances to the magnetic field, so as to modify their orientation and/or to move them, taking place prior or subsequently to the application of the third composition but before the compounds X and Y have completed their interaction.

9. Method according to claim 1, comprising at least:
the application to the said support of at least one first cosmetic composition comprising, in a physiologically acceptable medium, at least one compound X, respectively at least one compound Y, followed by
the application to the first composition of at least one second cosmetic composition comprising, in a physiologically acceptable medium, said magnetic substances, and
the application to the second composition of at least one third cosmetic composition comprising, in a physiologically acceptable medium, at least one compound Y, respectively at least one compound X, said second composition being capable of allowing the compounds X and Y to interact, with at least one of the compositions comprising, additionally at least one catalyst.

10. Method according to claim 9, the exposure of the magnetic substances to the magnetic field, so as to modify their orientation and/or to move them, taking place prior or subsequent to the application of the third composition but before the compounds X and Y have completed their interaction.

11. Method according to claim 1, X being selected from silicone compounds comprising at least two unsaturated aliphatic groups.

12. Method according to claim 11, in which compound X is a polyorganosiloxane comprising a silicone main chain whose unsaturated aliphatic groups are pendent from the main chain (side group) or located at the ends of the main chain of the compound (end group).

13. (canceled)

14. Method according to claim 1, compound X being selected from the polyorganosiloxanes comprising at least two unsaturated aliphatic groups each attached to a silicon atom.

15. Method according to claim 1, compound X being selected from the polyorganosiloxanes containing siloxane units of formula:
$${R}_{m} {R}^{′} {SiO}_{( \frac{3 - m}{2} )} \qquad ( I )$$
in which:
R represents a linear or cyclic, monovalent hydrocarbon group, having from 1 to 30 carbon atoms,
m is equal to 1 or 2 and
R′ represents:
an unsaturated aliphatic hydrocarbon group having from 2 to 10, preferably from 3 to 5 carbon atoms or
an unsaturated cyclic hydrocarbon group having from 5 to 8 carbon atoms.

16. Method according to claim 14, in which the polyorganosiloxane of formula (I) is such that R′ represents a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom.

17. (canceled)

18. Method according to claim 12, the polyorganosiloxanes additionally comprising units of formula:
$${R}_{n} {SiO}_{( \frac{4 - n}{2} )} \qquad ( II )$$
in which R represents a linear or cyclic, monovalent hydrocarbon group, having from 1 to 30 carbon atoms, and n is equal to 1, 2 or 3.

19. (canceled)

20. Method according to claim 1, in which compound Y comprises at least two free Si—H groups.

21. Method according to claim 1, compound Y being selected from the polyorganosiloxanes comprising at least one alkylhydrogenosiloxane unit with the following formula:
$${R}_{p} {HSiO}_{( \frac{3 - p}{2} )} \qquad ( III )$$
in which:
R represents a linear or cyclic, monovalent hydrocarbon group, having from 1 to 30 carbon atoms or a phenyl group, and p is equal to 1 or 2.

22. (canceled)

23. Method according to claim 17, in which Y is a polyorganosiloxane comprising at least two alkylhydrogenosiloxane units of formula —(H₃C)(H)Si—O— and optionally containing units —(H₃C)₂SiO—.

24.-25. (canceled)

26. Method according to claim 1, compound X being a dimethylsiloxane with vinylic end groups and compound Y being a polymethylhydrogenosiloxane.

27.-51. (canceled)
